(19) United States
(12) Patent Application Publication
(10) Pub. No.: US 2012/0220546 A1
(43) Pub. Date: Aug. 30, 2012

(54) COMPOSTIONS DESIGNED FOR THE INHIBITION AND/OR BLOCKING OF THE EPITHELIAL/MESENCHYMAL TRANSITION

(76) Inventors: Matías Antonio Avila Zaragoza, Pamplona (Navarra) (ES); Jesús Maria Bañales Asurmendi, Pamplona (Navarra) (ES); Maria Carmen Berasain Lasarte, Pamplona (Navarra) (ES); Fernando José Corrales Izquierdo, Pamplona (Navarra) (ES); MarÍa del Carmen Gil Puig, Pamplona (Navarra) (ES); María Ujue Latasa Sada, Pamplona (Navarra) (ES); Jon Lecanda Cordero, Pamplona (Navarra) (ES); Jesús Maria Prieto Valtueña, Pamplona (Navarra) (ES); Carlos Manuel Rodríguez Ortigosa, Pamplona (Navarra) (ES)

(21) Appl. No.: 13/462,991

(22) Filed: May 3, 2012

Related U.S. Application Data
(63) Continuation of application No. PCT/ES2010/070706, filed on Oct. 29, 2010.

(30) Foreign Application Priority Data
Nov. 5, 2009 (ES) P200930952

(51) Int. Cl. A61K 31/7076; A61P 1/16; A61P 35/00; C07H 19/167

(52) U.S. Cl. 514/46; 536/27.6

(57) ABSTRACT

5′-methylthioadenosine (MTA) is described as a compound that is susceptible to inhibiting and/or blocking the epithelial-mesenchymal transition (EMT), a process whereby epithelial cells become mesenchymal cells.

The periodical intake of MTA [every 24 h for 21 days] significantly improves fibrosis and the markers for hepatic cellular damage in KO-Mdr2 mice with MTA (28 mg/kg). Following the daily oral administration of MTA, both the expression of EMT markers in the total liver and appreciable signs of fibrosis are significantly reduced, indicating the beneficial effect of MTA on the liver affected by the lack of Mdr2. MTA is proposed to be a safe drug, suitable for oral formulation and without secondary effects, to be used in the prevention and/or treatment of diseases associated with EMT, including chronic cholestatic diseases, fibrosis and cholangiocarcinoma. On the other hand, MTA is proposed for application in anti-tumor therapies by inhibiting or blocking the EMT properties of CSC cells, in order to improve the prognosis of tumor development and the malignancy thereof.

### FIELD OF THE INVENTION

In general, this invention relates to compounds designed for the inhibition and/or blocking of the epithelial-mesenchymal transition (EMT) and for the prevention and/or treatment of diseases associated with EMT.

### BACKGROUND OF THE INVENTION

The epithelial-mesenchymal transition (EMT) is the process whereby epithelial cells become mesenchymal cells. EMT is a generally reversible and complex transdifferentiation of cells, characterized by the loss of cellular adhesion and a concurrent increase in cellular motility. The process begins with the repression of the expression of E-cadherin, the breakage of intercellular bonds and the loss of the apicobasal polarity typical of epithelial cells (cells that are arranged in the form of a honeycomb, with perfectly formed intercellular adhesive bonds), which are transformed into mesenchymal cells, acquiring new migration, invasiveness and fibrogenic functional properties.

The EMT is a characteristic feature of proliferating cells, being essential for a wide number of development processes, including formation of the mesoderm and the neural tube. Although the EMT may be induced under culture in most epithelial cells with a wide range of stimuli, this process only occurs in vivo during embryogenesis and it seems that it also mediates certain pathological conditions, such as carcinoma and fibrotic processes. Well-known regulators of EMT are TGFβ₁ (inducer) and BMP-7 (inhibitor) (Xu et al. Nephrol 2009; 22(3): 403-10).

The activation of the EMT program has been proposed as a critical mechanism in the acquisition of the malignant phenotype of cancerous epithelial cells. The EMT is the first step in the metastatic chain, whereby cancerous epithelial cells leave the primary nodule, contributing to the spreading of the tumor. By blocking the EMT of cancerous epithelial cells, one may expect a better prognosis in the development of the tumor and the metastases thereof.

Jointly with the activation of resident fibroblasts and the recruitment of fibrocytes derived from the bone marrow, EMT transdifferentiation has also been identified as a pathogenic mechanism that promotes fibrosis in various organs, such as the lung (EMT of alveolar epithelial cells), kidney (EMT of tubular epithelial cells), intestine (Crohn's disease), eye (cataracts, pterigium) or liver (Kisseleva and Brenner, Experimental Biology and Medicine 2008; 233: 109-122).

Thus, for example, there is increasing evidence to confirm that the epithelial cells of the hepatic parenchyma may acquire a malignant phenotype with EMT characteristics in vivo (Gressner et al., Comparative Hepatology 2007; 6:7). In particular, it has been shown that cells associated with the biliary tree acquire mesenchymal properties and contribute to the development of fibrosis in the course of chronic cholestatic diseases, such as primary biliary cirrhosis (PBC) or primary sclerosing cholangitis (PSC).

The histology of PSC exhibits properties similar to those of autoimmune hepatitis and evolves with a particular symptomatology of inflammation and fibrosis in the ducts of the intrahepatic and extrahepatic biliary tract, which produces the narrowing and obstruction thereof. As a result, there is a change in the content of biliary secretion—with a different conjugation of salts and pH changes—, which may induce a reaction of the cholangiocyte phenotype, characterised by the production of various pro-inflammatory and pro-fibrogenic cytokines and chemokines. Furthermore, PSC represents the most common risk factor in the pathogenesis of cholangiocarcinoma, an epithelial tumor that represents the second most frequent type of liver cancer. Most patients with PSC develop cholangiocarcinoma within 30 months following the initial diagnosis, and surgery is the only effective healing alternative to increase these patients' survival. The absence of adequate biliary or serum markers makes it impossible to monitor the progression from PSC to cholangiocarcinoma and, although different drug combinations have been tested, currently there is no suitable treatment available to stop the progression of PSC. The various medical treatments tested in this regard include anti-fibrogenic agents such as colchicine, cupruretic agents such as D-penicillamine, or immunosuppressants such as corticoids, azathioprine, methotrexate or cyclosporin. None of them has proven efficacy on the evolution of the disease or on survival. High doses of ursodeoxycholic acid have shown beneficial effects on the analytical evolution, but the histology of bile duct cells remains unaltered (LaRusso et al., Hepatology 2006; 44: 746-764).

Recent studies on neoplastic tissues have made it possible to identify cell populations with stem cell properties in the tumors; these cells have been called cancer stem cells or CSCs. Although these cells may represent less than 0.01% of the total number of cells, some researchers suggest that all tumor cells exclusively originate from this particular type of cells. The involvement of these CSC cells in tumorogenic processes may explain the greater malignancy and worse prognosis of certain carcinomas. Recently, the participation of CSC cells has been related to the origin and development of cancer due to their EMT properties. The induction of the EMT in cells from normal or neoplastic mammary tissues has shown an enrichment of their stem cell properties (Mani et al. Cell 2008; 133: 704-15). The modulation of the EMT process makes it possible to confer their main cellular characteristics to CSCs, which causes a greater aggressiveness in the tumor. Therefore, it has been suggested that the specific control or blocking of the EMT in the CSC cells of certain carcinomas will allow for an improved prognosis in the development of the tumor and the malignancy thereof.

Due to the heterogeneity of tumor formations, the currently used anti-tumour therapies are oriented toward eliminating the cancer stem cells, CSCs, or preventing the appearance thereof. However, CSCs have been identified as the main agents responsible for the recurrence or resistance to conventional chemotherapeutic agents experienced by patients with some types of cancers. Therapies with the classic cytostatic agents (taxols, platinum compounds, etc.) and radiotherapy do not eliminate cells with EMT properties; for this reason, due to their acquired cellular properties, CSC cells are resistant to most current chemotherapeutic treatments.

Since the EMT is an underlying mechanism common to some types of fibrosis and cancer, it may be stated that currently there are no commercially available medicaments that specifically regulate it and which may be used for the prevention and/or treatment of diseases associated with this process. However, the development of molecules capable of inhibiting and/or blocking the EMT has recently been proposed as an anti-tumor therapeutic strategy in cancerous and fibrotic processes.

Some strategies reported in the state of the art for modulation of the EMT comprise the use of lipocalin 2 (WO2006/078717) or regulators of the protein related to Golgi-associated pathogenesis (GARP-1) (WO2007038264).

WO2007/069839 relates to the use of erythropoietin (EPO) in the preparation of an agent designed to inhibit the EMT and to prevent or treat fibrosis. Moreover, it describes a prevention and treatment method for fibrosis using EPO, a protein capable of inhibiting the TGFβ₁-induced EMT. However, since EPO receptors are formed on the surface of most tumor cells, there is a possibility that some EPO preparations may stimulate the growth of said cells.

US2006234911 relates to a pharmaceutical composition that comprises a quantity of a kinase inhibitor capable of reversing the EMT (selected from a TGFβ₁ kinase, a RhoA kinase or a p38-MAP kinase inhibitor). It also relates to a method of reversing the EMT transition in a patient with a fibrotic disease or cancer.

Given the wide variety of cellular processes wherein kinases and TGFβ₁ participate, the use of inhibitors may affect healthy cells and interfere with other cellular processes that are essential for appropriate cellular growth. Consequently, these compounds exhibit various secondary effects and have a very limited tolerance. Moreover, it must be considered that the treated patients may develop mutations in the molecular target that needs to be inhibited. Thus, undesireable resistance phenomena arise and there are limited therapeutic alternatives. Last, but not least, this type of molecular targets is specific for each type of pathology, and thus may be irrelevant in a given type of cancer.

Now that the relationship between the EMT mechanism and the origin and development of tumors is known, it may be stated that currently there are no commercially available chemotherapeutic drugs that make it possible to specifically regulate the EMT of CSC cells in tumor tissues. In fact, most radiotherapy or chemotherapy protocols use anti-tumor drugs that do not affect the EMT, such as, for example: taxols, gemcitabine, cisplatin, oxaliplatin, etc. This leads to cases where neither chemotherapy nor radiation treatment completely eradicate the disease, such that a residual cell population with a high presence of CSCs remains. Therefore, the development of new drugs capable of inhibiting and/or blocking the EMT in CSC cells has recently been proposed as an additional therapeutic strategy in anti-cancer treatments based on conventional chemotherapeutic agents.

Gupta et al. describe a treatment with salinomycin that is capable of inhibiting, in vivo, the growth of tumor cells in mice with breast cancer and inducing an increase in the epithelial differentiation of their tumor cells. This study makes it possible to identify agents with a specific toxicity toward breast CSC cells, despite the difficulty in identifying these cells in tumorogenic populations and their relative instability in cell cultures.

For all these reasons, an objective of this invention is to provide a medicament capable of reversing or inhibiting the EMT which is safe, suitable for oral formulations and without secondary effects.

Another objective of this invention is to provide a medicament capable of inhibiting or blocking the EMT which is suitable to prevent and/or treat fibrotic diseases associated with said EMT.

Another objective of this invention is to establish a new pharmacological therapy designed to improve the symptoms and stop the progression of chronic cholestatic diseases, including fibrosis and the establishment of cholangiocarcinoma.

Another objective of this invention is to provide a compound capable of controlling cancer by acting on the EMT, a step that initiates the metastatic cascade. Moreover, another objective of this invention is to provide a suitable compound for application in anti-tumor therapies as an adjuvant or additional treatment to the administration of conventional chemotherapeutic agents, inhibiting or blocking the EMT properties of CSC cells.

### SUMMARY DESCRIPTION OF THE INVENTION

5′-methylthioadenosine (MTA) is a lipophilic adenine nucleoside that contains sulfur, produced from S-adenosylmethionine (SAM) during the synthesis of the polyamines spermine and spermidine. MTA is a suitable drug for oral formulations due to its small size and its hydrophilic character, and does not have the secondary effects that other methyl-transferase inhibitors have. In previous studies, it has been administered, in an acute and chronic manner, to animal models of liver damage and systemic inflammation, and has shown efficacy and a good safety profile, with an ID50 of 2.9+0.4 g/kg (i.m.) in rats. In humans, MTA is also well tolerated. It has been tested in 28 healthy subjects (21-48 years of age) with an MTA dose of 100 mg every 8 hours for 3 days and with a dose of 600 mg per day for 1 month without signs of toxicity. In an effort to explore the potential of MTA, it has been surprisingly observed that sustained concentrations of this molecule are sufficient to inhibit the EMT both in vitro and in vivo (zebrafish model and murine model of PSC).

Thus, this invention relates to MTA, its pharmaceutically acceptable salts and/or the prodrugs thereof, for use in the inhibition and/or blocking of the EMT. In other words, this invention relates to the use of MTA, its pharmaceutically acceptable salts and/or the prodrugs thereof in the preparation of a medicament useful for inhibiting and/or blocking the EMT. Similarly, this invention relates to a method for the inhibition and/or blocking of the EMT which comprises administering a therapeutically effective quantity of MTA, its pharmaceutically acceptable salts and/or the prodrugs thereof to a subject who needs it.

This invention also relates to a pharmaceutical composition that comprises: a) MTA and/or its pharmaceutically acceptable salts and/or the prodrugs thereof, and b) a pharmaceutically acceptable excipient, for use in the inhibition and/or blocking of the EMT. In other words, this invention relates to the use of a pharmaceutical composition that comprises: a) MTA and/or its pharmaceutically acceptable salts and/or the prodrugs thereof, and b) a pharmaceutically acceptable excipient, in the preparation of a medicament useful for inhibiting and/or blocking the EMT. Moreover, the invention relates to a method for the inhibition and/or blocking of the EMT which comprises administering, to a subject in need thereof, a therapeutically effective quantity of a pharmaceutical composition that contains: a) said MTA, its pharmaceutically acceptable salts and/or prodrugs, and b) a pharmaceutically acceptable excipient.

Similarly, this invention also relates to MTA, its pharmaceutically acceptable salts and/or the prodrugs thereof, for use in the prevention and/or treatment of a chronic cholestatic disease. In other words, this invention also relates to the use of MTA, its pharmaceutically acceptable salts and/or the prodrugs thereof in the preparation of a medicament for the prevention and/or treatment of a chronic cholestatic disease. Similarly, this invention also relates to a method for the prevention and/or treatment of a chronic cholestatic disease which comprises administering an effective quantity of MTA, its pharmaceutically acceptable salts and/or the prodrugs thereof to a subject who needs it.

Moreover, this invention also relates to MTA, its pharmaceutically acceptable salts and/or the prodrugs thereof, for use in a therapy protocol for inhibit and/or block the EMT, characterised in that it is an adjuvant or additional treatment to the administration of the conventional chemotherapeutic agents used in clinical practise.

### BRIEF DESCRIPTION OF THE FIGURES

FIG. 1. The EMT induced in vitro in AML-12 cells and cholangiocytes in the presence of TGFβ₁ is prevented by the addition of MTA. a) Control. b) 200 μM MTA. c) 500 μM MTA. d) 80 pM TGFβ₁. e) TGFβ₁ 80 pM+200 μM MTA. f) TGFβ₁ 80 pM+500 μM MTA. Representative photographs show the morphological transformation of epithelial cells following the addition of TGFβ₁ in hepatic AML-12 cells (A), and WT cholangiocytes (B), mice cholangiocytes treated with 80 pM TGFβ₁ and a combination of 80 pM TGFβ₁ and 500 μM MTA during 24 hours (a,b,c) and 48 hours (d,e,f) (C).

FIG. 2. MTA prevents the migration of epithelial cells that develop the EMT in the presence of TGFβ₁. A. Representative Photographs show the transformations observed in NMuMG cells in the Scratch Assay. An incision is made prior to the beginning of the treatments, and the migration that occurs following the different specified treatments with TGFβ₁ and/or MTA is measured. Panels: a) Control (carrier,

DMSO). b) 200 μM MTA. c) 500 μM MTA. d) 80 pM TGFβ₁. e) 80 pM TGFβ₁+200 μM MTA. f) 80 pM TGFβ₁+500 μM MTA.

Following the same technique, the migration is also analyzed in cholangiocytes from KO-Mdr2 mice (C).

FIG. 3. The expression of the characteristic EMT markers is reversed by incubation with MTA. The graphs show the results of the relative expression of different markers in EMT models in the presence of TGFβ₁ and/or MTA, as compared to the expression obtained in non-stimulated control cells. They show the changes in the expression of specific EMT markers following the application of increasing doses of MTA in three independent experiments. A. AML-12 cells. B. WT cholangiocytes. (Note: AU, arbitrary units).

FIG. 4. Effects of the MTA on the inhibition of the TGFβ₁-dependent EMT signalling. The activation of the signalling pathway is shown in A. AML-12 cells that were stimulated with TGFβ₁ and MTA, with the specified concentrations. B. Primary cholangiocytes isolated from healthy mice treated with TGFβ₁ and MTA.

FIG. 5. Effects of the MTA during the embryonary development of zebrafish and analysis of the effects associated with the epithelial-mesenchymal transition, EMT.

5A. Zebrafish embryos, 96 hpf, injected with 500 μM MTA in the blood stream at 24 hpf. Panels A, C, E, G, phase contrast; panels B, D, F, H, GFP. A, B embryo with a normal development. The arrow in panels C, E and G shows the existence of pericardial edema of a different magnitude which correlates with the intensity of the effect shown. The asterisk in the same images shows the area where the blood cells accumulate. The following components of the circulatory system are indicated in panel B: IS, intersegmental vessel; DA, dorsal artery; PCV, posterior cardinal vein. The yellow line in panels D and F shows the area where the PCV disappears. In panel H, the PCV has completely disappeared from the embryo, whereas part of the DA and the ISs has also disappeared.

5B. Representative figures that show the morphological details related to the effects of MTA on the appearance of pericardial edema, accumulation of blood cells in the tail and tubular hearts. Also shown is the direct effect related to the atrophy of the heart valves due to inhibition of the EMT (Timmerman et al. Genes Dev 2004; 18: 99-115).

5C. 72-hpf embryos which show the effect of MTA on the binding of somites. Panel A shows a control embryo with a correct formation of the somites. Some embryos treated with 1 mM MTA show an adequate formation, despite the presence of problems in the development of the vascular system (panel B), whereas others clearly exhibit disorganised somites in the anterior area of the trunk (panel C). The arrows show examples of perfectly formed somite bonds. The bracket indicates the area of blood cell accumulation. The key shows the area where the somites are most clearly disorganised.

5D. Summary-table of the phenotypes induced by the addition of MTA. The numbers in each box represent the number of embryos that exhibited the specified phenotype.

FIG. 6. Effects of MTA in an in vivo model of Primary Sclerosing Cholangitis (PSC). Administration of MTA (28 mg/kg, every 24 h) was performed for 21 days on control mice (WT) and Mdr2 knockout mice (KO-Mdr2). A. Hepatomegaly in the KO-Mdr2 mice decreases following the administration of MTA. B. Quantification by real-time RT-PCR of the EMT markers following the administration of MTA (28 mg/kg) for 21 days. The expression of EMT-initiating genes in the liver of the KO-Mdr2 mice increases as compared to the healthy mice. Following treatment with MTA, the expression of markers indicative of EMT in the liver is reduced.

FIG. 7. Effects of MTA following a prolonged 21-day treatment: WT and KO-Mdr2 mice, comparing those untreated vs treated with MTA (28 mg/kg, every 24 h). A. Fibrosis in the KO-Mdr2 mice is reversed following the administration of MTA. The figure shows the deposition of extracellular collagen by Sirius Red staining. B. Quantification of the serum levels of marker enzymes for liver damage. C. Decrease in ductular proliferation. Staining of proliferation marker Ki67 in non-parenchymatous hepatic cells and positive cell count, shown by immunohistochemistry. D. Decrease in α-SMA staining following treatment with MTA in KO-Mdr2 mice, shown by immunohistochemistry and real-time RT-PCR. E. Representative Figure of the immunohistochemistry in front of Tenascin C, where it is showed an increment in the dye surrounding the portal area in KO-Mdr2 (panel b) compared to the WT mice (Mdr2+/+) (panel a). Said expression diminishes to the present levels in WT mice when KO-Mdr2 are treated with MTA (panel c).

FIG. 8. Characteristics CsCs (Cancer Stem Cells) markers in WT and KO-Mdr2 cholangiocytes. A. CK19, Snail, Vimentin, and Tenascin C expression determined by conventional PCR. B. CK19, Snail, Vimentin, and Tenascin C expression determined by real time PCR. It is observed a decrease in CK19 expression (epithelial marker) and an increase in mesenchymal markers associated to CSC presence (Snaill, Vimentin, and Tenascin C) in KO-Mdr2 cholangiocytes.

FIG. 9. The treatment with MTA reverses the TGFβ₁ effect as EMT inducer over the E-cadherin expression in cholangiocytes. Representative figures of the E-cadherin expression determined by immunofluorescence in WT cholangiocytes (A) and KO-Mdr2 cholangiocytes (B). It is observed that the treatment with TGFβ₁ decreases the expression of the E-cadherin protein, while the treatment with 500 μM MTA reverses it to its normal state.

FIG. 10. The MTA treatment reverses the TGFβ effect as EMT inducer over the TGFβ expression and secretion in cholangiocytes. A. TGFβ secretion determined by western blot. In the figure it can be observed how, in samples of conditioned medium, only the cholangiocytes from KO-Mdr2 mice secrete TGFβ to the medium. In addition, the MTA is able to inhibit such secretion after 48 hours of treatment. B. In cholangiocytes cell extracts from WT mice, TGFβ expression is induced after a 48 hour treatment with TGFβ₁. The combined treatment with MTA and TGFβ₁ decreases the protein expression of the growth factor.

FIG. 11. The MTA treatment reverses the TGFβ₁ effect as EMT inducer over the collagen expression and secretion in cholangiocytes. A representative figure is showed of the collagen secretion, determined by western blot, in samples obtained from the culture medium of KO-Mdr2 cholangiocytes. It can be observed how the collagen and the pro-collagen are increased after the treatment with TGFβ₁ and how the MTA reverse such effect.

### DESCRIPTION OF THE INVENTION

A first aspect of this invention relates to MTA, its pharmaceutically acceptable salts and/or the prodrugs thereof, for use in the inhibition and/or blocking of the EMT.

MTA, which is also referred to herein as 5′-methylthioadenosine, is a commercial product that may be supplied, for example, by the Sigma company. Alternatively, this compound may be obtained by methods known to those skilled in the art, for example, from S-adenosyl-methionine (SAM), following the method described by Schlenk F. et al. (Arch Bioch Biophys 964; 106: 95-100). The CAS registry number of MTA is 2457-80-9, and its structural formula is:

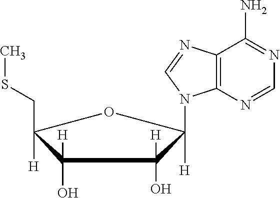

The term “salt”, as mentioned in this invention, is intended to comprise any stable salt that MTA is capable of forming The pharmaceutically acceptable salts are those preferred. Those salts that are not pharmaceutically acceptable are also included within the scope of this invention, since they relate to intermediates that may be useful in the preparation of compounds with pharmacological activity.

The salts may be obtained, in a practical manner, by treating the basic form of MTA with said appropriate acids, such as inorganic acids, such as hydrazides, for example, hydrochloric or hydrobromic acid, sulfuric acid, nitric acid, phosphoric acid, and similar acids; or organic acids such as, for example, acetic, propanoic, hydroxyacetic, lactic, pyruvic, oxalic (that is, ethanedioic), malonic, succinic (that is, butanedioic acid), maleic, fumaric, malic (that is, hydroxybutanedioic acid), tartaric, citric, methanesulfonic, ethanesulfonic, benzenesulfonic, p-toluenesulfonic, cyclamic, salicylic, p-aminosalicylic, pamoic and similar acids.

The pharmaceutically acceptable salts may be obtained by treating the basic form of MTA with said appropriate pharmaceutically acceptable acids, such as inorganic acids, for example, hydrazides, including hydrochloric, hydrobromic and similar acids; sulfuric acid; nitric acid; phosphoric acid and similar acids; or organic acids, for example, acetic, propanoic, hydroxyacetic, 2-hydroxypropanoic, 2-oxopropanoic, oxalic, malonic, succinic, maleic, fumaric, malic, tartaric, 2-hydroxy-1,2,3-propane-tricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic or other similar acids. Inversely, the salt form may be converted into the free basic form by treatment with alkali.

The term “pharmaceutically acceptable” means that a compound or combination of compounds is sufficiently compatible with the other components in a formulation, and is not harmful for the patient to the levels accepted by industry standards.

For therapeutic use, the 5′-methylthioadenosine salts are those wherein the counter ion is pharmaceutically acceptable.

The term “prodrug”, as used in this invention, includes any compound derived from MTA, for example, ester, amide, phosphate, etc., which, after being administered to a subject, is capable of providing MTA or the pharmaceutically acceptable salt thereof, directly or indirectly, to said subject. Preferably, said derivative is a compound that increases the bioavailability of MTA when it is administered to a subject or induces the release of MTA in a biological compartment. The nature of said derivative is not critical, provided that it may be administered to a subject and that it provides MTA in a biological compartment of the subject. The preparation of said prodrug may be performed by conventional methods known to those skilled in the art. The prodrugs of MTA may be prepared in a practical manner, for example, by binding a progroup to one or both hydroxyl groups in the ribose ring. One example of a prodrug of MTA is 2′-[(2Z)-3-(4-hydroxyphenyl)-2-methoxy-2-propenoate]-3′-[(2E)-3-(1H-imidazol-4-yl)-2-propenoate]-5′-S-methyl-5′-thio-adenosine (Kehraus et al., J Med Chem 2004; 47(9): 2243-2255). Another example of a prodrug or precursor of MTA is S-adenosylmethionine (SAM).

The term “EMT” refers to the cellular reprogramming process whereby fully differentiated epithelial cells adopt the molecular and phenotypic characteristics of mesenchymal cells.

The term “inhibiting the EMT”, as used in this invention, refers to preventing, repressing, suspending the epithelial-mesenchymal transition, transitorily or permanently, by the action of an adequate stimulus. The term “inhibition of the EMT” refers to the act of inhibiting the EMT, as “inhibiting the EMT” is defined immediately above.

The term “blocking the EMT” refers to stopping the epithelial-mesenchymal transition in any of its stages. In this invention, it also refers to hindering or hampering said epithelial-mesenchymal transition by the action of an adequate stimulus. The term “blocking of the EMT” refers to the act of blocking the EMT, as “blocking the EMT” is defined immediately above.

In a particular embodiment, this invention relates to MTA, its pharmaceutically acceptable salts and/or the prodrugs thereof, for use in the inhibition and/or blocking of the TGFβ₁-dependent EMT.

As explained above, TGFβ₁ is the main stimulator of the EMT.

In this invention, the researchers have shown that MTA is particularly useful to reverse EMT markers induced by this cytokine.

“Transdifferentiation” entails the conversion of a cell to another cell type from a different lineage, accompanied by the loss of specific markers and the function of the original cell type, and the acquisition of markers and the function from the other cell type. As the term is used in this invention, transdifferentiation refers to the conversion of an epithelial cell into a mesenchymal cell. In adult epithelial tissue, the increase in the number of transdifferentiated cells by EMT-dependent mechanisms favours the progression of tumours and other pathological processes, such as fibrosis.

Thus, in another particular embodiment, this invention relates to MTA, its pharmaceutically acceptable salts and/or the prodrugs thereof, for use in the inhibition and/or blocking of the EMT, both TGF-β-dependent or independent, in the prevention and/or treatment of a disease associated with said EMT. In this invention, the terms “associated with”, “mediated by” and “related to” are used interchangeably and refer to diseases that evolve with an EMT process of epithelial cells as one of their pathogenic bases.

The term “preventing” refers to avoiding the occurrence, the existence or, alternatively, delaying the appearance or reappearance of a disease, disorder or condition whereto said term is applied, or of one or more symptoms associated with a disease, disorder or condition. The term “prevention” refers to the act of preventing, as “preventing” is defined immediately above.

The term “treating”, as used in this invention, refers to reversing, relieving or inhibiting the progression of the disorder or condition whereto said term is applied, or one or more symptoms of said disorders or conditions. The term “treatment” refers to the act of treating, as “treating” is defined immediately above.

In an embodiment of this invention, MTA, its pharmaceutically acceptable salts and/or the prodrugs thereof may be used in the inhibition and/or blocking of the EMT for the prevention and/or treatment of a pathological condition related to the EMT, as an adjuvant or additional treatment following a radiotherapy or chemotherapy treatment. In this invention, the term “adjuvant or additional treatment” refers to a treatment that accompanies or is subsequent to a previous treatment that is considered to be the main treatment. It also refers to a medical treatment of neoplastic diseases that is complementary to another that has been previously performed, including chemotherapy, radiotherapy treatments or hormone therapy, used to eliminate the cancerous cells that may remain following a surgery.

The term “radiotherapy”, as used in this invention, refers to a medical treatment of neoplastic diseases that uses ionising radiation (X-rays or radioactivity, which includes gamma rays and alpha particles) to eliminate the cancerous cells, by means of a local treatment. Radiotherapy acts on the tumor, destroying the malignant cells, and thus prevents them from growing and reproducing. This action may also be exerted on normal tissues; however, tumor tissues are more sensitive to radiation and cannot repair the damage caused as efficiently as normal tissues, such that they are destroyed by blocking the cell cycle.

The term “chemotherapy” refers to a medical treatment of neoplastic diseases based on the administration of drugs whose function it is to prevent the reproduction of the cancerous cells. Said drugs are called cytostatic drugs, or cytostatic or cytotoxic agents. The action mechanism of chemotherapy is to cause a cellular alteration, either in the synthesis of nucleic acids, cell division or protein synthesis. The action of the different cytotoxic or cytostatic drugs varies depending on the dose that is administered. Due to their unspecificity, they affect other normal cells and tissues in the body, particularly if they are undergoing active division. Therefore, chemotherapy is the use of various drugs which have the property of interfering with the cell cycle, causing the destruction of cells.

Thus, in another particular embodiment, this invention relates to MTA, its pharmaceutically acceptable salts and/or the prodrugs thereof, for use in the elimination of CSCs in subjects who undergo recurrence in the face of conventional chemotherapeutic agents.

The term “CSCs” (Cancer Stem Cells) refers to cancer stem cells, which are specific tumor cells that have the capacity to regenerate the malignant phenotype. They are cancerous cells that may be found in solid tumors and haematological cancers, have characteristics typical of normal stem cells; specifically, they have the capacity to generate any cell typology pertaining to a particular cancer sample. Therefore, CSCs are considered to be tumorigenic or tumor-initiating cells, they may generate tumours through two main properties typical of stem cells: differentiation (they are able to lead to the heterogeneity of cell types present in the tumor) and self-renewal (they are able to give rise to new stem cells with the same properties). CSCs are different from normal stem cells in that they exhibit an unbalanced differentiation and self-renewal processes; they also lose the regulation patterns of normal proliferation. However, like normal stem cells, CSCs are capable of bearing adverse conditions that affect the tissue environment.

The term “cells with EMT properties” refers to cells that lack epithelial properties (cellular adhesion, polarity, loss of motility and migration) and have characteristics of mesenchymal cells (fibroblast phenotype with a loss of expression of epithelial markers) with the corresponding markers; greater motility and migration.

The term “recurrence” refers to the reappearance of the tumor mass in a subject, following the regression of the tumor obtained with a radiotherapy therapy and/or a chemotherapy treatment with conventional chemotherapeutic agents.

The term “conventional chemotherapeutic agents” refers to the cytotoxic or anti-tumor drugs routinely used in clinical protocols (taxols, gemcitabine, cisplatin, oxaliplatin, 5-FU, etc.).

In an embodiment of this invention, MTA, its pharmaceutically acceptable salts and/or the prodrugs thereof may be used in the inhibition and/or blocking of the EMT for the prevention and/or treatment of a pathological condition related to the EMT, regardless of the cause. Preferably, said pathological condition is selected from epithelial cancer, EMT-mediated fibrosis or cholestatic disease.

The term “epithelial cancer”, as applied in this invention, is synonymous with carcinoma and refers to the malignant neoplasms that originate in cell strains of an epithelial or glandular origin. In a particular embodiment of the invention, MTA, its pharmaceutically acceptable salts and/or the prodrugs thereof may be used in the inhibition and/or blocking of the EMT for the prevention and/or treatment of a carcinoma, selected, for example, from: adenocarcinoma (bronchiolo-alveolar adenocarcinoma, clear-cell adenocarcinoma, folicular adenocarcinoma, mucinous adenocarcinoma, papillary adenocarcinoma, en cuirasse adenocarcinoma, sebaceous adenocarcinoma, adrenocortical adenocarcinoma, carcinoid tumor, acinar cell carcinoma, adenoid cystic carcinoma, ductal carcinoma, endometroid carcinoma, pancreatic adenocarcinoma, gastric carcinoma, colorectal cancer, hepatocellular carcinoma, non-infiltrating intraductal carcinoma, islet cell carcinoma, lobular carcinoma, mucoepidermoid carcinoma, neuroendocrine carcinoma, renal cell carcinoma, signet ring cell carcinoma, cutaneous appendage carcinoma, cholangiocarcinoma, choriocarcinoma, cystadenocarcinoma, Klatskin tumour, extramammary Paget's disease, adenosquamous carcinoma; basal cell carcinoma, basosquamous carcinoma; Ehrlich tumour carcinoma; giant cell carcinoma; in situ carcinoma (cervical intraepithelial neoplasia and prostatic intraepithelial neoplasia; Krebs 2 carcinoma; large cell carcinoma; Lewis lung carcinoma; non-small cell lung carcinoma; papillary carcinoma; squamous cell carcinoma (Bowen's disease); transitional cell carcinoma; warty carcinoma. Other examples are adnexal and cutaneous appendage neoplasms, such as sebaceous adenocarcinomas, cutaneous appendage carcinoma; basal cell neoplasms, such as basal cell carcinomas (basal cell nevus syndrome), basosquamous carcinoma and pilomatrixoma; cystic, mucinous and serous neoplasms, such as mucinous adenocarcinomas, mucoepidermoid carcinoma, signet ring cell carcinoma/Krukenberg tumor), cystadenocarcinoma (mucinous cystadenocarcinoma, papillary cystadenocarcinoma, serous cystadenocarcinoma), cystadenoma (mucinous cystadenoma, papillary cystadenoma, serous cystadenoma), mucoepidermoid tumour and peritoneal pseudomixoma, ductal, lobular and medullary neoplasms, such as ductal carcinoma (mammary ductal carcinoma, pancreatic ductal carcinoma), non-infiltrating intraductal carcinoma (mammary Paget's disease), lobular carcinoma, medullary carcinoma, extramammary Paget's disease, intraductal papiloma; fibroepithelial neoplasms, such as adenofibroma, Brenner tumour; mesothelial neoplasms, such as adenomatoid tumor, mesothelioma (cystic mesothelioma); and squamous cell neoplasms, such as acanthoma, papillary carcinoma, squamous cell carcinoma (Bowen's disease), warty carcinoma, papiloma (inverted papiloma).

It also seems reasonable to inhibit the molecular and cellular processes that contribute to the dissemination of cancer to other tissues through metastasis. This is an extraordinarily complex process that develops in a sequential manner. First, the malignant cells might undergo an EMT process and are then able to detach from the primary nodule, to cross the extracellular matrix, the endothelium and other cell structures. Subsequently, these cells must survive in blood circulation to finally settle in a different physiological niche, where they will form a metastatic focus. These stages, including the EMT transdifferentiation, are potentially susceptible to therapeutic manipulation, since each of these mechanisms contributes to the aggressiveness and the malignant spreading. In this invention, MTA, its pharmaceutically acceptable salts and/or the prodrugs thereof are especially useful in the inhibition and/or blocking of the EMT in order to prevent the development of metastasis in a subject with epithelial cancer. The term “subject” means animals, such as dogs, cats, cows, horses, sheep and humans. The particularly preferred subjects are mammals, including humans of both sexes.

The term “fibrosis” refers to the excess formation or development of fibrous connective tissue in an organ or tissue as a consequence of a repair or reactive process, characterised by an increase in the production and deposition of extracellular matrix. As used in this invention, the term fibrosis refers to those fibrotic processes associated with an EMT transdifferentiation towards myofibroblasts.

In a particular embodiment of the invention, MTA, its pharmaceutically acceptable salts and/or the prodrugs thereof may be used in the inhibition and/or blocking of the EMT for the prevention and/or treatment of a fibrotic disease associated with EMT, such as, for example: idiopathic pulmonary fibrosis, epithelial fibrosis (e.g. scleroderma, post-traumatic or post-surgery scarring), ocular fibrosis (e.g. ocular sclerosis, conjunctival or cornea scarring, pterigium), pancreatic fibrosis, pulmonary fibrosis, cardiac fibrosis (e.g. endomyocardial fibrosis, idiopathic myocardiopathy), hepatic fibrosis (e.g. cirrhosis, steatosis), intestinal fibrosis, progressive massive fibrosis, proliferative fibrosis, neoplastic fibrosis and others.

The term “cholestasia” or “cholestasis” refers to the biliary secretory failure that is caused by a functional alteration of biliary secretion at the level of the hepatocytes (hepatocellular cholestasia) or of a functional or obstructive alteration at the level of the intra- and extra-hepatic biliary ducts or conducts (ductal or conductal cholestasis).

Thus, in another particular embodiment of the invention, MTA, its pharmaceutically acceptable salts and/or the prodrugs thereof may be used in the inhibition and/or blocking of the EMT for the prevention and/or treatment of a cholestatic disease, for example, progressive familial intrahepatic cholestasis (PFIC), benign recurrent intrahepatic cholestasis (BRIC), primary biliary cirrhosis (PBC), primary sclerosing cholangitis (PSC), autoimmune cholangitis, biliary atresia, adult idiopathic ductopenia, graft rejection, graft against host disease (EICH), cholestasis of pregnancy, cholangiocarcinoma or bile duct cancer, Klastki tumour and others. In a preferred particular embodiment of the invention, MTA, its pharmaceutically acceptable salts and/or the prodrugs thereof are used in the inhibition and/or blocking of the EMT for the prevention and/or treatment of a chronic cholestatic disease.

In a more preferred particular embodiment, MTA, its pharmaceutically acceptable salts and/or the prodrugs thereof are used in the inhibition and/or blocking of the EMT for the prevention and/or treatment of PSC or PBC.

In another, also preferred, particular embodiment, MTA, its pharmaceutically acceptable salts and/or the prodrugs thereof are used in the inhibition and/or blocking of the EMT for the prevention and/or treatment of cholangiocarcinoma.

The various uses and methods that use MTA, its pharmaceutically acceptable salts and/or the prodrugs thereof in this invention comprise acute administration, that is, the administration which takes place several minutes to approximately several hours after the lesion, or chronic administration, suitable for chronic disorders.

In the uses and methods designed for the inhibition and/or blocking of the EMT by the administration of MTA, its pharmaceutically acceptable salts and/or the prodrugs thereof, these compounds may be used as a first line or initial therapy to prevent and/or treat a disease associated with said EMT. Alternatively, MTA, its pharmaceutically acceptable salts and/or the prodrugs thereof may be used as an adjuvant or as an additional therapy to other drugs.

Therefore, in another embodiment of this invention, in the uses and methods designed for the inhibition and/or blocking of the EMT, MTA, its pharmaceutically acceptable salts and/or the prodrugs thereof are used as adjuvants or additional therapy in a subject with a fibrotic, cancerous and/or cholestatic disease who is being treated with one or more anti-fibrotic, anticancer and/or anti-cholestatic compounds.

A second aspect of this invention relates to a pharmaceutical composition, hereinafter pharmaceutical composition of the invention, which comprises: a) MTA and/or its pharmaceutically acceptable salts and/or the prodrugs thereof, and b) a pharmaceutically acceptable excipient, for use in the inhibition and/or blocking of the EMT. In a particular embodiment of the invention, the latter relates to a pharmaceutical composition of the invention, as defined immediately above, for use in the inhibition and/or blocking of the EMT to prevent and/or treat a disease associated with said EMT.

The diseases that may be prevented and/or treated include all those indicated when describing the uses of MTA.

MTA, its pharmaceutically acceptable salts and/or the prodrugs thereof may be formulated in several pharmaceutical forms for administration purposes. The appropriate compositions include all the compositions habitually used for the systematic administration of drugs, for example, any solid composition (for example, tablets, capsules, granules, etc.) or liquid composition (for example, solutions, suspensions, emulsions, etc.). In order to prepare the pharmaceutical compositions of MTA, an effective quantity of MTA, optionally in the form of a salt or a prodrug, as the active principle, is combined in an intimate mixture with a pharmaceutically acceptable carrier, which may adopt a wide variety of forms depending on the preparation form desired for administration. These pharmaceutical compositions are desirable in the form of suitable unit doses, particularly for oral, rectal, percutaneous, intrathecal, intravenous route or parenteral injection. For example, when preparing the compositions for oral dosage, any of the habitual pharmaceutical media may be used, such as, for example, water, glycols, oils, alcohols and similar media in the case of liquid oral preparations, such as suspensions, syrups, elixirs, emulsions and solutions; or solid carriers, such as starches, sugars, kaolin, lubricants, bonding agents, disintegrating agents and similar agents in the case of powders, pills, capsules and tablets. Due to their ease of administration, tablets and capsules represent the most advantageous oral unit dosage forms, in which case solid pharmaceutical carriers are obviously used. In the case of parenteral compositions, the carrier habitually comprises sterile water, at least for the most part, although other ingredients may be included, for example, to increase the solubility. Injectable solutions may be prepared where, for example, the carrier comprises saline solution, glucose solution or a mixture of saline solution and glucose solution. Injectable suspensions may also be prepared, in which case appropriate liquid carriers, suspension agents and similar agents may be used. Solid-form preparations are also included, which are intended to be converted into liquid-form preparations shortly before they are to be used. In compositions suitable for percutaneous administration, the carrier optionally comprises a suitable penetration-enhancing agent or a wetting agent, or both, optionally combined with suitable additives of any type in smaller proportions; these additives do not introduce a significant harmful effect on the skin. A review of the different pharmaceutical forms for the administration of drugs and the preparation thereof may be found in the book “Tratado de Farmacia Galénica”, by C. Faulí and Trillo, 10th Edition, 1993, Luzán 5, S. A. de Ediciones.

It is especially advantageous to formulate the pharmaceutical compositions mentioned above in the form of unit doses in order to facilitate the administration and uniformity of the dosage. The unit dosage form, as used in this invention, refers to suitable physically discrete units as unit dosages, each unit containing a pre-determined quantity of active principle calculated to produce the desired therapeutic effect associated with the required pharmaceutical carrier. Examples of said forms of unit dosages are tablets (including grooved or coated tablets), capsules, pills, suppositories, powder packets, wafers, injectable solutions or suspensions and similar forms, and segregated multiples thereof.

The compositions in accordance with this invention, including the unit dosage forms, may contain the active principle in a quantity within the range of approximately 0.1% to 70%, or approximately 0.5% to 50%, or approximately 1% to 25%, or approximately 5% to 20%, the rest comprising the carrier, where the preceding percentages are in p/p over the total weight of the composition or dosage form.

The dose of MTA, its pharmaceutically acceptable salts and/or the prodrugs thereof to be administered depends on each individual case and, as is habitual, must be adapted to the individual case conditions for an optimal effect. Therefore, it naturally depends on the frequency of administration and the potency and duration of the action of the compound used in each case for therapy or prophylaxis, but also on the nature and the severity of the disease and the symptoms, and the sex, age, weight, co-medication and individual sensitivity of the subject to be treated, and on whether the therapy is acute or prophylactic. The doses may be adapted as a function of the weight and for pediatric applications. An “effective quantity” of MTA and the pharmaceutically acceptable salts thereof may be, for example, within the range between 0.01 mg and 50 g per day, 0.02 mg and 40 g per day, 0.05 mg and 30 g per day, 0.1 mg and 20 g per day, 0.2 mg and 10 g per day, 0.5 mg and 5 g per day, 1 mg and 3 g per day, 2 mg and 2 g per day, 5 mg and 1.5 g per day, 10 mg and 1 g per day, 10 mg and 500 mg per day.

The daily doses may be administered q.d. or in multiple quantities, such as b.i.d., t.i.d. or q.i.d.

A third aspect of the invention relates to the use of MTA, its pharmaceutically acceptable salts and/or the prodrugs thereof, or to the use of a pharmaceutical composition of the invention, in the preparation of a medicament useful for inhibiting and/or blocking the EMT. In a particular embodiment, said medicament is useful for inhibiting and/or blocking the EMT in the prevention and/or treatment of a disease associated with said EMT and, in particular, those indicated above.

Similarly, this invention relates to a method for the inhibition and/or blocking of the EMT, which comprises administering a therapeutically effective quantity of MTA, its pharmaceutically acceptable salts and/or the prodrugs thereof, or of an effective quantity of the pharmaceutical composition of the invention, to a subject in need thereof. In a particular embodiment, said method is useful to prevent and/or treat a disease associated with EMT, in particular, the diseases described above.

The inhibitory properties of the epithelial-mesenchymal transition exhibited by MTA, its pharmaceutically acceptable salts and/or the prodrugs thereof cause as a consequence the prevention or the partial or total treatment of different alterations characteristic of cholestatic diseases caused by the EMT in epithelial cells. Therefore, this invention also relates to MTA, its pharmaceutically acceptable salts and/or the prodrugs thereof, for use in the prevention and/or treatment of a cholestatic disease. In other words, this invention also relates to the use of MTA, its pharmaceutically acceptable salts and/or the prodrugs thereof in the preparation of a medicament for the prevention and/or treatment of a cholestatic disease. Similarly, this invention also relates to a method designed for the prevention and/or treatment of a cholestatic disease, which comprises administering an effective quantity of MTA, its pharmaceutically acceptable salts and/or the prodrugs thereof to a subject who needs it. The cholestatic disease may be any of the diseases mentioned above, although preferably it is selected from PSC, PBC or cholangiocarcinoma.

The invention is described below by means of examples that are illustrative of the invention, but are not intended to limit it.

### EXAMPLES

The invention is described below by means of examples that are illustrative of the invention, but are not intended to limit it.

Example 1

### The EMT is Prevented in the Presence of MTA

Epithelial cells under culture exhibit an organized morphology wherein intercellular adhesive bonds may be observed between the cells. Overall, a perfectly defined monolayer is appreciated, formed by cuboidal- or hexagonal-type cells. When they develop the EMT, the cells show an elongated or fusiform morphology—characteristic of fibroblasts—with a disorganized appearance and accompanied by the loss of intercellular bonds.

A. Methods

Cell culture. The cell lines were obtained from ATCC and cultured under the recommended conditions. The AML-12 cells (mouse hepatocytes) were grown in DMEM/F12-glutamax, with a 1× antibiotic solution (penicillin-streptomycin, Invitrogen), dexamethasone (40 ng/ml, Sigma-Aldrich), 1×'Insulin-Transferrin-Selenium (Invitrogen), in 10% fetal bovine serum (FBS, Hyclone).

Isolation, purification and culturing of primary cholangiocytes. In parallel, cholangiocytes from normal mice (wild-type; Mdr2+/+) and mice deficient in the Mdr2 biliary transport gene (knockout; Mdr2−/−), the so-called WT and KO-Mdr2 mice, respectively, were obtained. The mice were anaesthesized prior to in situ perfusion of the liver. First, a washing was performed with Swim's S-77 medium, with penicillin/streptomycin, containing BSA, insulin and heparin. Subsequently, collagenase type I was added, and perfusion was continued for 10 minutes. In order to isolate the biliary tree, the liver was placed on a plate with culture medium, removing the capsule, and the hepatocytes were eliminated. The biliary ducts were incubated with collagenase and dispase in order to eliminate the hepatic parenchymatous cells. The resulting biliary tree was disaggregated and re-suspended in culture medium with hyaluronidase. The medium containing the cells was filtered, and the cholangiocytes were retained in the filter. These were re-suspended in culture medium in order to seed them in a flask over a base of rigid collagen in Ham's F10 culture medium supplemented with bovine hypophysis extract, glutamine, penicillin/streptomycin.

Stimulation with TGFβ₁ and/or MTA. The lyophilized MTA was dissolved in DMSO (Sigma). The cells were seeded in 6-well plates, 300,000 cells in each. After 24 h had elapsed, MTA was added, at concentrations of 200-500 μM, to these monolayer cell cultures, using DMSO as the control carrier. After 3 h of incubation, the human TGFβ₁ recombinant protein (R&D Systems) was added, at a concentration of 80 pM, for the indicated periods of time. In order to stimulate the cholangiocytes, these were cultured in commercial plates (BD Bioscience) treated with collagen type I (Invitrogen).

B. Results

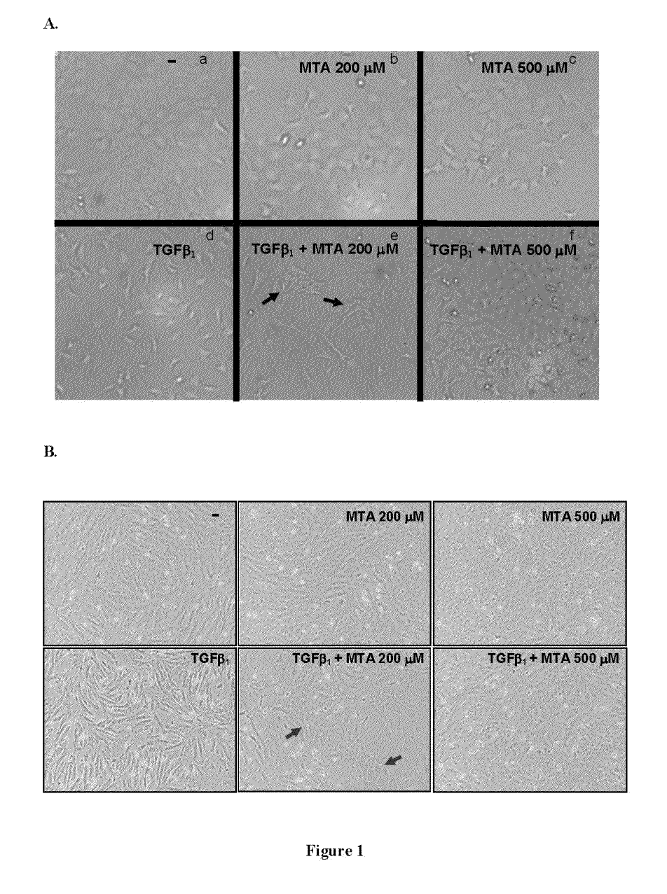

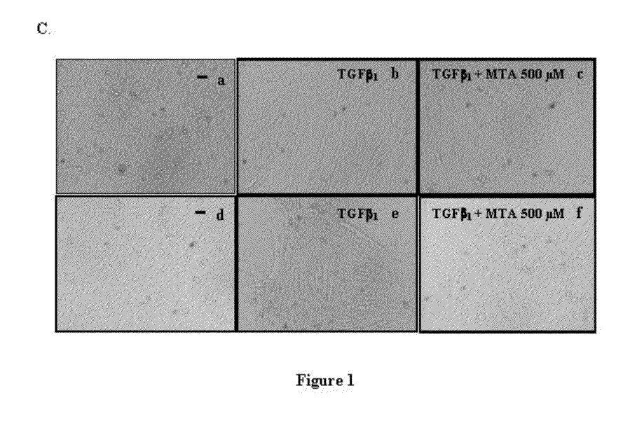

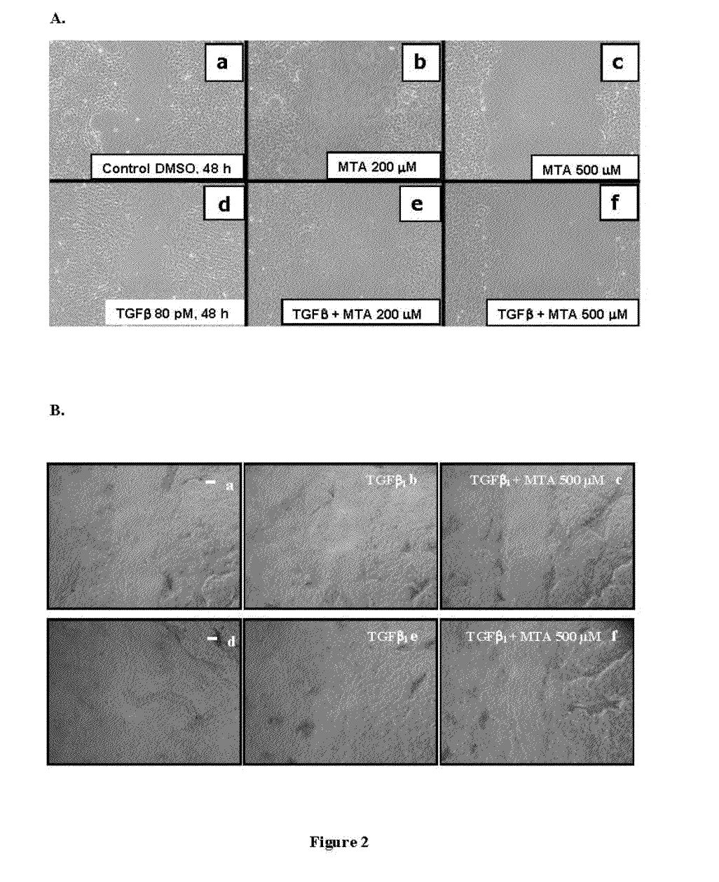

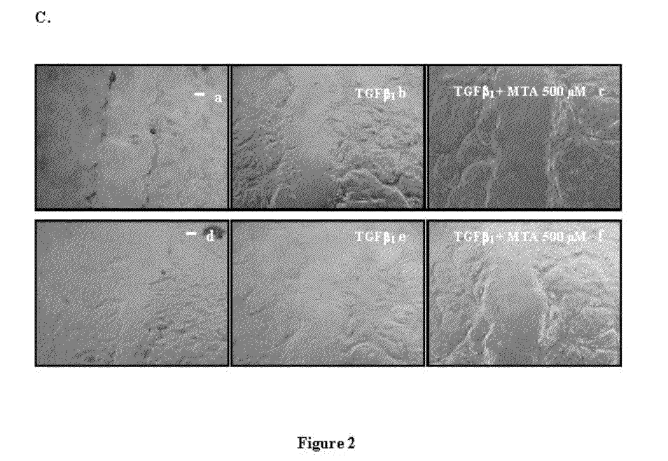

TGFβ₁ is capable of inducing a cellular phenotype associated with the EMT (FIG. 1), with the appearance of a new morphology of stellar cells, as shown in panel d. In the presence of MTA, the cells maintain the epithelial phenotype even when stimulation of the cells with TGFβ₁ continues (panel e-f). The inhibition of the TGFβ₁-dependent EMT following the addition of MTA is observed in both cell types, in both the AML-12 hepatocytes (FIG. 1A) and the WT mouse primary cholangiocytes (FIG. 1B). In this cell type it is observed, in both 24 and 48 hours, the TGFβ₁ effect over the epithelial cells, and how after the MTA treatment with a 500 μM dose reverses the fibroblastic phenotype (FIG. 1C).

### Example 2

### MTA Prevents the Migration of Epithelial Cells Associated with the EMT

The Scratch Assay is performed in confluent cells. Similar incisions are made in each of the wells by means of a tip that moves across the diameter of the circular well. The treatments (carrier, TGFβ₁, MTA) are performed following the incision. Every 24 hours, the morphology and the migration capacity—sealing of the fissure by the presence of cells—are observed in each of the wells. Representative photographs of at least two independent experiments after 24-48 hours from the beginning of the excision and application of the treatments are shown.

A. Methods

Cell culture. The cell lines were obtained from ATCC and cultured under the manufacturer's conditions. The NMuMG cells (mouse mammary gland) were cultured in DMEM, 1× antibiotic solution (penicillin-streptomycin), 1× glutamine, 10% FBS, and supplemented with insulin (Sigma).

The cultured cholangiocytes were isolated from WT and KO-Mdr2 mice as described in example 1.

Migration experiment. Scratch assays (n=3) were performed, wherein a scratch is made on confluent cells with a plastic tip. After washing the NMuMG cells with PBS, serum-free medium (or medium with 5% serum: results not shown) was added, and the treatment with MTA/TGFβ₁ was started, as specified above (FIG. 2A). The photographs shown were taken 24-48 h after the beginning of the treatments.

The cholangiocytes obtained from WT mice (FIG. 2B) and KO-Mdr2 (FIG. 2C) were grown in complete medium and after the scratch was made it was analyzed the migration at 24 h (panels a, b, c) and 48 h (panels d, e, and f) under 80 pM TGFβ₁ and 500 μM MTA treatments.

B. Results

TGFβ₁ is capable of inducing the EMT, by colonising the free space in the cells (FIG. 2) (panel d). The phenotype and the migration induced by TGFβ₁ are partially reversed in the presence of 200 μM MTA (panel e). The simultaneous addition of MTA (500 μM) prevents the EMT: the phenotype is completely epithelial following incubation with 500 μM MTA, and no migration of cells is observed in the jagged space (panel f). The same effect was observed in both WT and KO-Mdr2 cholangiocytes (FIG. 2B).

20

### Example 3

### Expression of the EMT Markers is Reversed by Means of Incubation with MTA

The EMT conditions the loss of the polarity of cells and a functional transformation thereof, with a significant reduction in the adhesive properties of cells and the consequent de novo expression of numerous fibroblastic markers. As a result, an increase in the motility and invasiveness of cells is induced via EMT, thereby facilitating that the cells disaggregate, migrate and cross the extracellular matrix.

A. Methods

The culturing of AML-12 cells and the obtainment of primary cholangiocytes were performed as described in example 1.

Quantification of the expression of the EMT markers. The total RNA was isolated following the Trizol method (Sigma). 2 μg of RNA were used to obtain the cDNA, and it was purified in Centrisep columns. The real-time PCR reaction conditions were performed using the IQ-SYBRGreen kit (BioRad), following the manufacturer's recommendations. The results show 3 independent experiments; the values of p<0.05 are considered to be significant differences.

B. Results

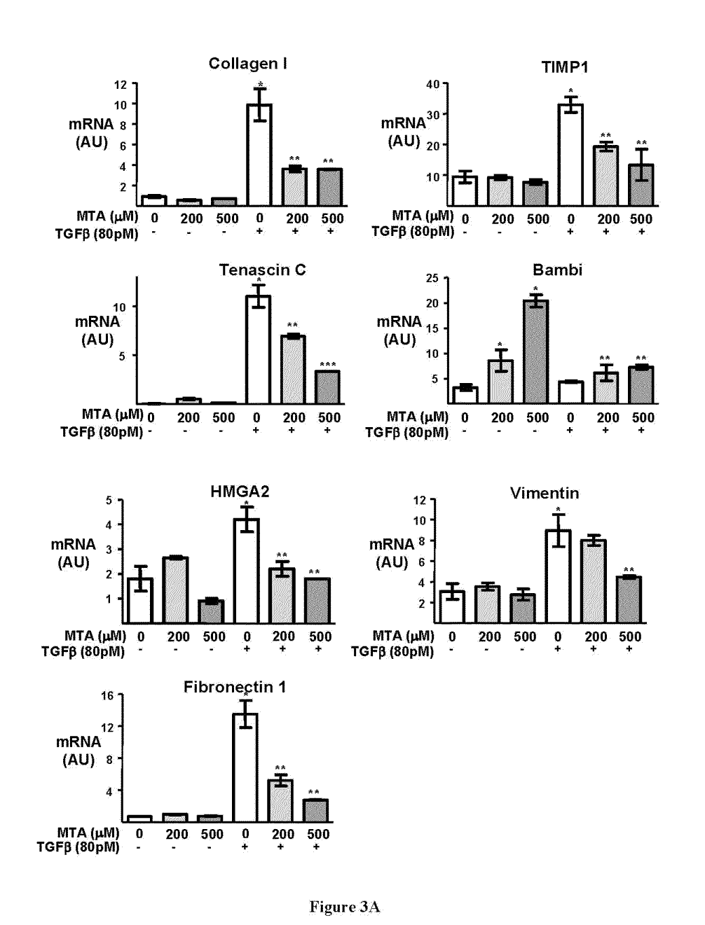

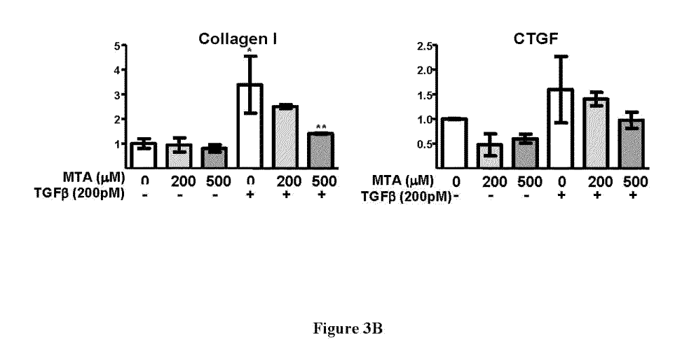

The effects of TGFβ₁ on the induction of collagen, TIMP-1, Tenascin C, HMGA2, Vimentin and Fibronectin 1 -markers with mesenchymatous properties—are reversed by MTA. The specific effect of some of these markers (e.g. TIMP1, Tenascin C or HMGA2) on the induction of the EMT has been shown in vitro. Moreover, the capacity to modulate the expression of the Bambi receptor, a negative regulator of the TGFβ₁ signalling pathway, has been observed in both AML12 (FIG. 3A) and WT mice cholangiocytes (FIG. 3B).

### Example 4

### Effects of MTA on the Inhibition of the TGFβ₁-dependent EMT Signalling

TGFβ₁ signals through the formation of a tetrameric complex of two transmembrane receptors (called TβRI and TβRII) with serine-threonine kinase activity. Briefly, the binding of TGFβ₁ to receptor TβRII leads to the phosphorylation of TβRI and the consequent activation of its kinase activity to phosphorylate Smad2 and/or Smad3 in the cytoplasm. The phosphorylation of these receptor-dependent Smads facilitates the binding thereof to Smad4. The Smads complex is then translocated to the nucleus in order to associate with other co-activators, co-repressors and DNA-binding proteins, binding to target gene promoter sequences and activating the complex EMT program. This canonical pathway may be supplemented with other signaling pathways also regulated by TGFβ, such as MAPK and Akt/PI3 kinases. The pro-oncogenic result via EMT depends on the cellular context and the integration of these different intracellular signaling pathways, but EMT mechanisms in tumoral cells are yet to be completely defined.

A. Methods

The culture of both AML-12 cells and the primary cholangiocytes were performed as described in example 1.

Western blot. The protein lysates were obtained in RIPA solution, supplemented with protease inhibitors (Sigma). Electrophoresis was performed starting with 10 μg of proteins in a 10% polyacrylamide gel, and transferred to a nitrocellulose membrane (Bio-Rad). The blocking was performed in a solution of 1% BSA (Santa Cruz)/1% powder milk/0.1% Tween-20/20 mM NaF, for 1 h at room temperature. The antibodies used -for 1 h- were diluted in the same solution using the following quantities: phospho-Smad3 (1:1,000, Calbiochem), phospho-Smad2 (1:1,000, Calbiochem), Smad2/3(1:1,000, Chemicon) and E-cadherin (1:20,000, BD Biosciences).

B. Results

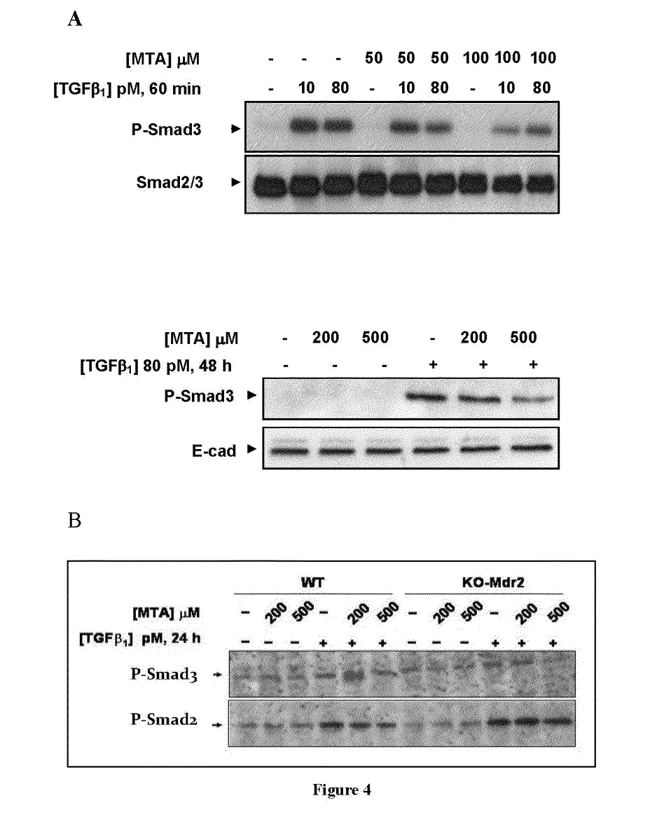

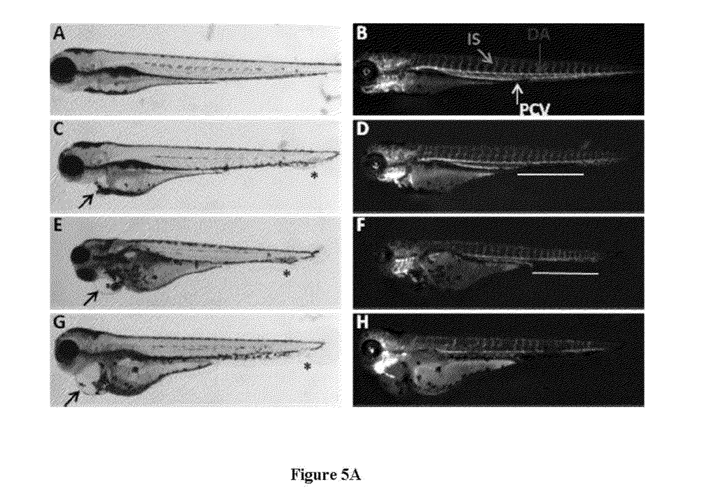

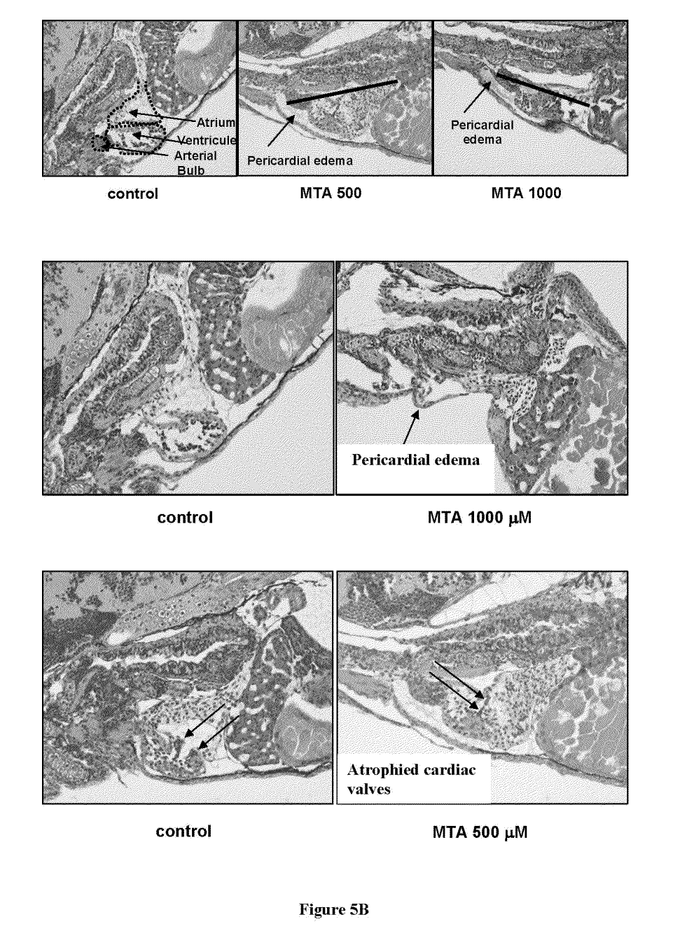

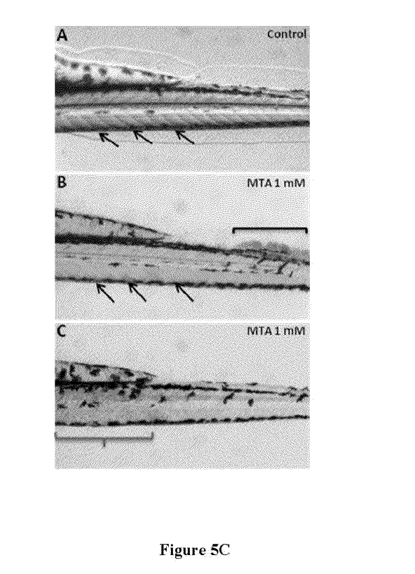

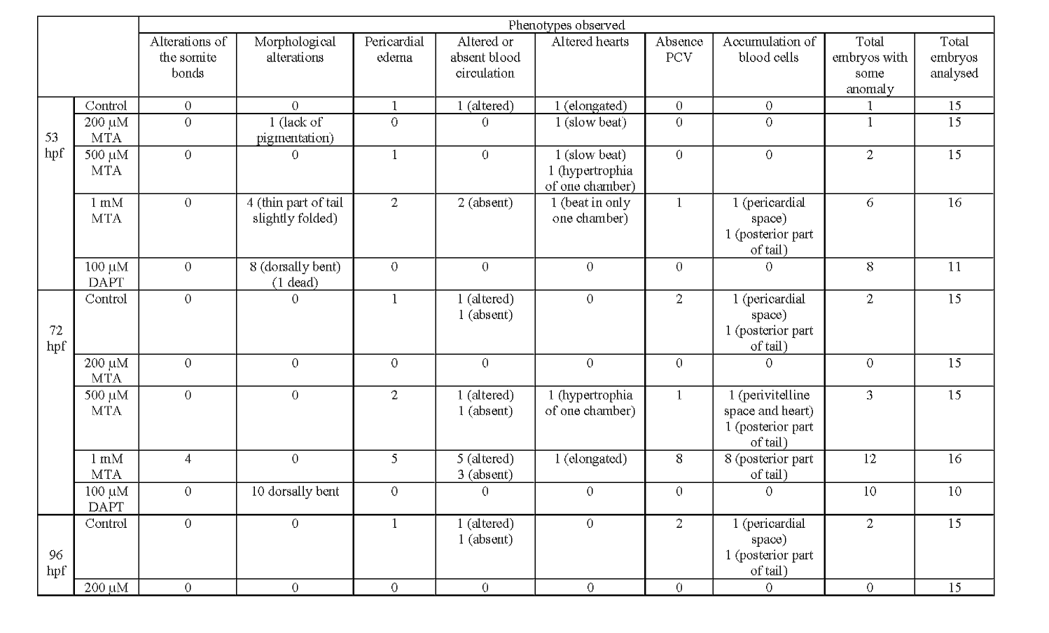

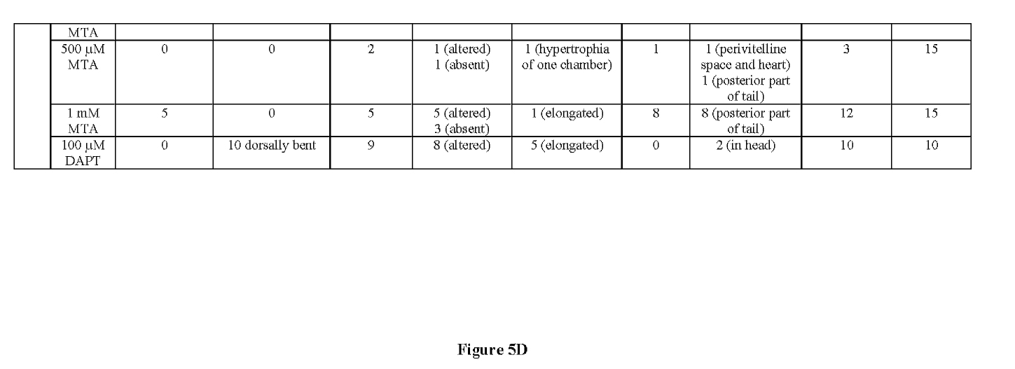

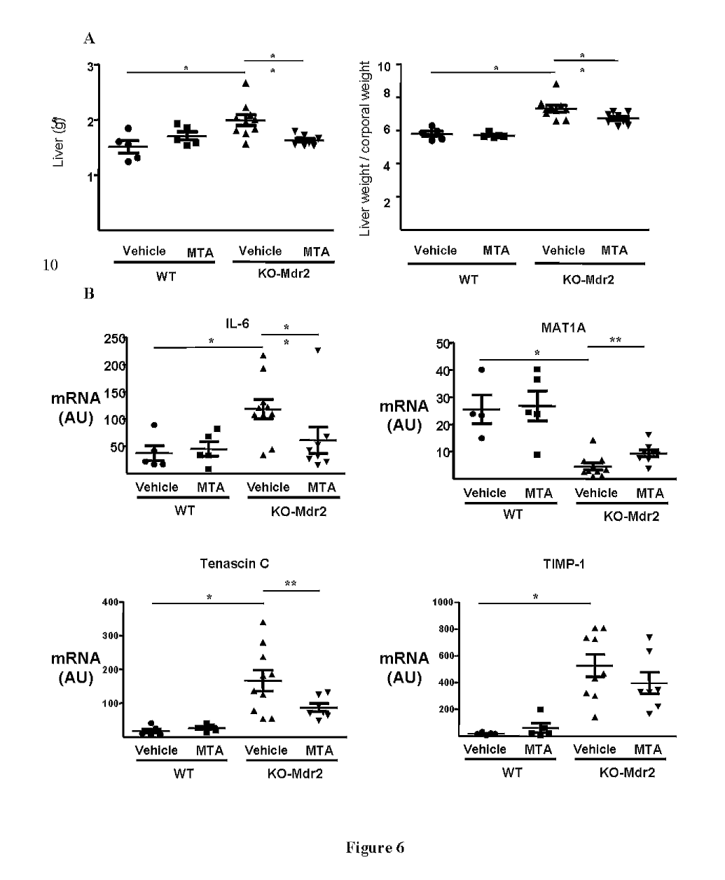

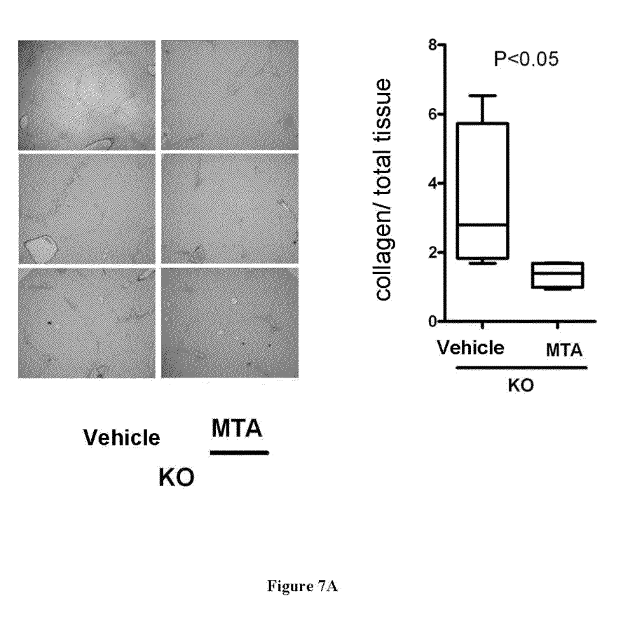

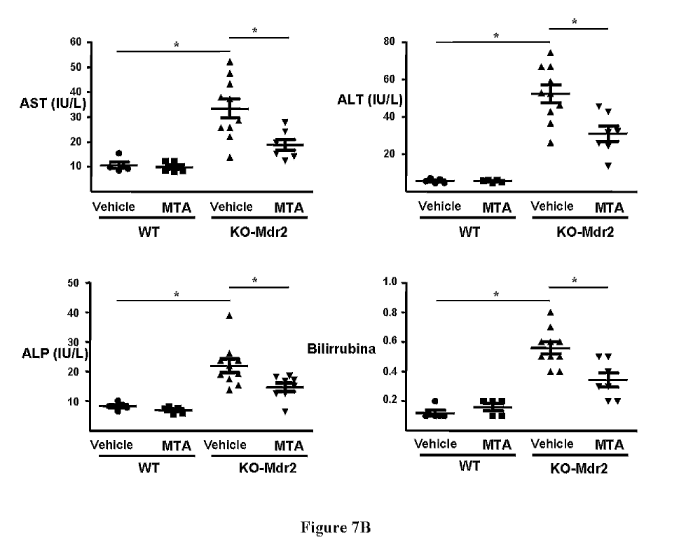

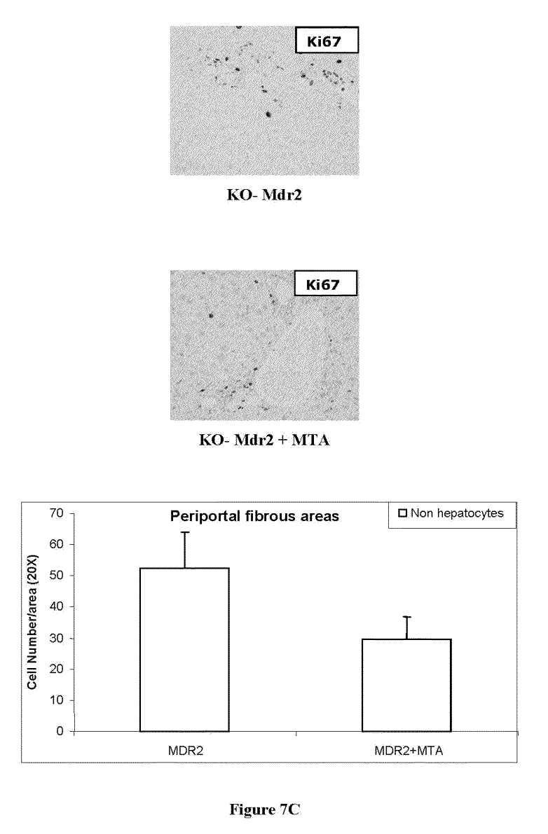

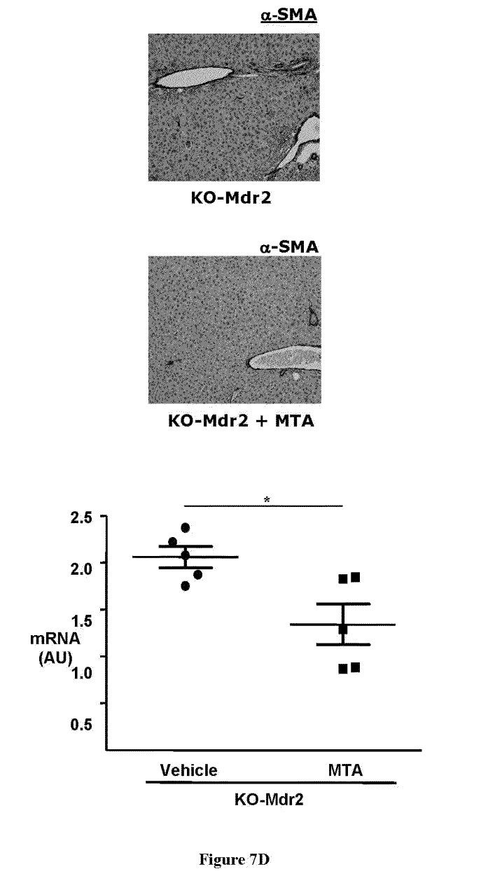

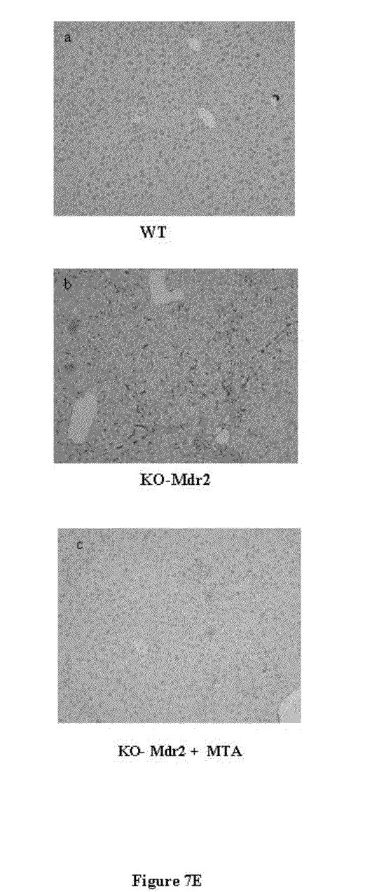

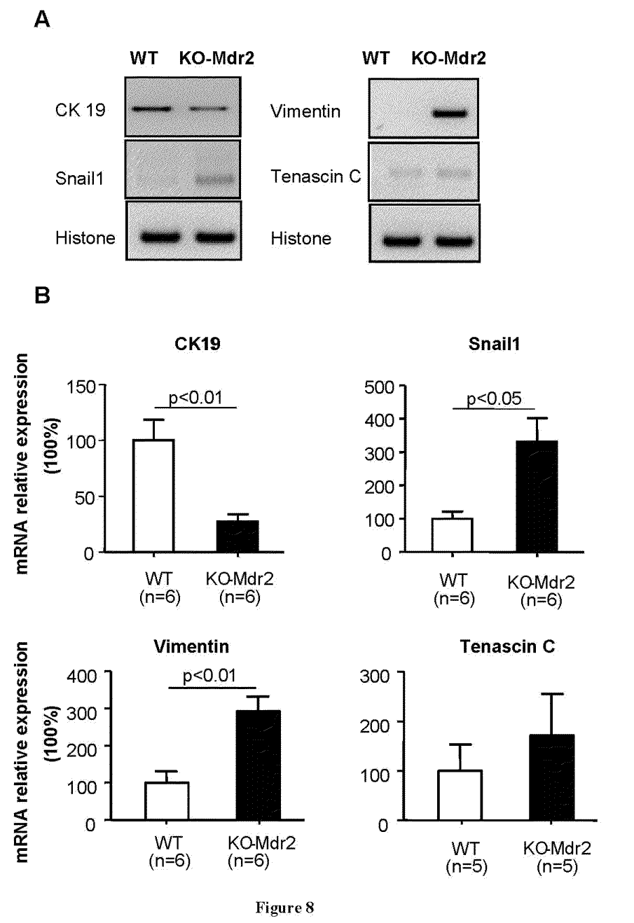

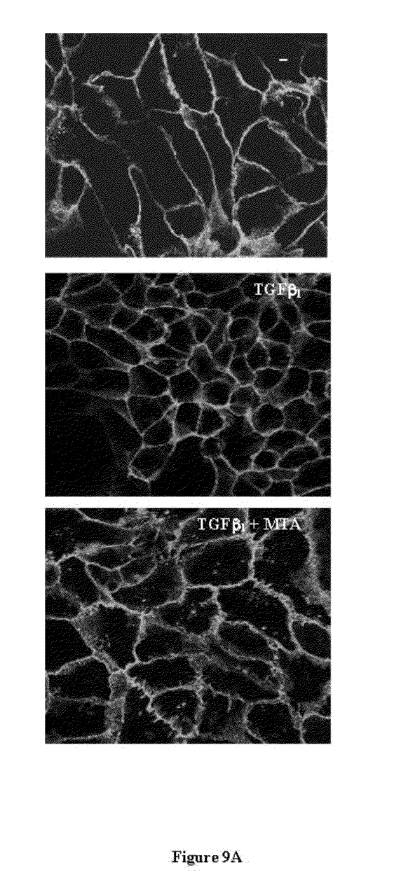

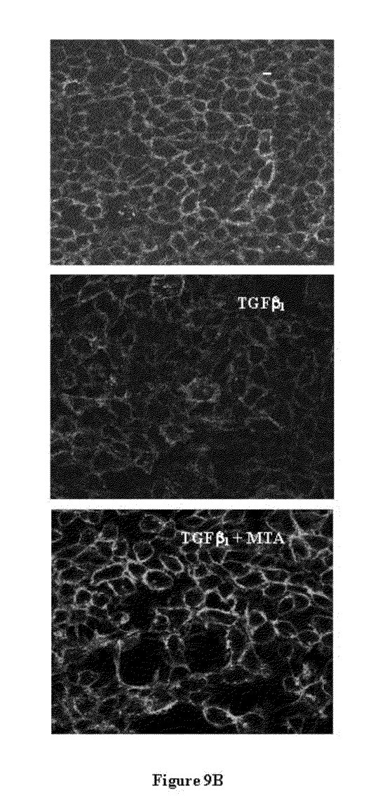

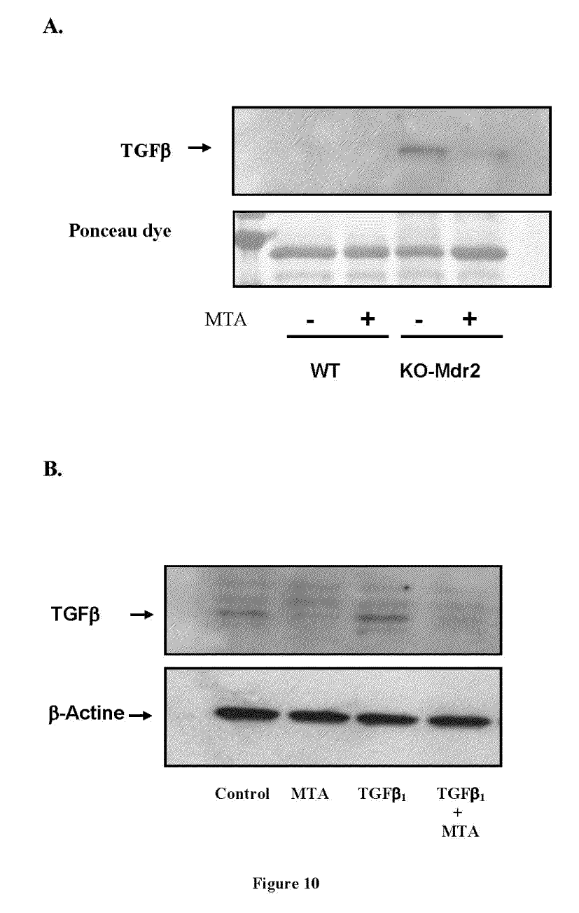

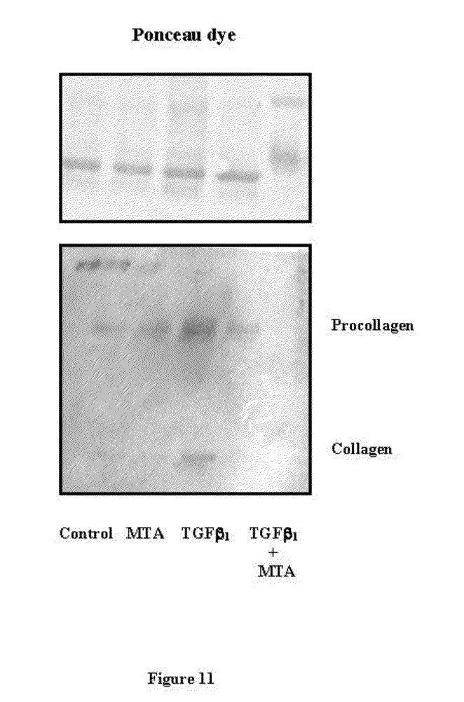

In both cell types, AML-12 hepatocytes (FIG. 4A) and primary cholangiocytes (FIG. 4B), it is observed that MTA is capable of inhibiting the direct effects of TGFβ₁ on the phosphorylation of the receptor-associated Smads (R-Smads), Smad2 and Smad3. From the inhibition of the activation of these signal transducer factors, it may be concluded that MTA is capable of specifically inhibiting signaling pathways initiated by TGFβ₁.

### Example 5

### Zebrafish Model

Various significant genes for the EMT in higher vertebrates are preserved in the zebrafish, where they have similar functions within this process. The zinc-carrier protein associated with LIV1 breast cancer controls the EMT during gastrulation in the zebrafish. The phosphatase PEZ protein is significant in the formation of various zebrafish organs, participating in the control of the EMT exerted by TGFβ₁. The family of Notch membrane receptors induces the epithelial-mesenchymal transition during cardiac development in both the zebrafish and in rodents. Therefore, the inhibitory effect of a compound on EMT may be studied in zebrafish embryos through its effect on processes wherein the epithelial-mesenchymal transition is essential (gastrulation, formation of somite bonds, cardiac development, etc.). In the zebrafish model, all this may be performed by direct observation, thanks to the embryos' transparency and simplicity and the existence of transgenic lines that allow for the visualisation of, for example, the heart or the vascular system. The use of zebrafish embryos is a system for the testing of compounds that combines the biological complexity of in vivo models with a low cost and a high-throughput capacity.

A. Methods

The necessary embryos to develop the study were obtained and kept at 28.5° C. in the incubator until the treatment was to begin (24 hpf and 32 hpf). The embryos were dechorionated at 24 hpf and deposited on a Petri dish with E3 medium. Prior to injecting the product, the fish were treated with 0.04% tricaine and, once they were asleep, they were placed on an agarose plate with a number of wedge-shaped grooves, where the embryos were aligned and immobilised in order to be injected. The mixture of the assay product to be injected was loaded in the injection capillary and a volume of 15 nl was injected in the perivitelline space. 20 embryos were injected per condition. After the injection, they were collected in a Petri dish and examined under a Zeiss stereoscope in order to select those that showed the injected mixture in the blood stream. These were deposited in 24-well plates (5 embryos per well) in 0.5 ml of E3 medium without methylene blue or antibiotic, and incubated at 28.5° C. until they were to be analyzed. As a control, embryos were injected with HBSS +0.5% rhodamine-dextran.

Moreover, a positive control of the inhibitory effect on the EMT was included in the assay, which consisted of 32-hpf embryos treated with 100 μM DAPT dissolved in the medium. 2.5 μl of a 20 mM stock solution were administered in a volume of 0.5 ml of E3 medium, in order to obtain a final concentration of 100 μM.

The embryos were observed at 53, 72 and 96 hpf under the Olympus microscope and/or a Zeiss stereoscope and the phenotypes observed were recorded. The embryos used to obtain representative images of the phenotypes observed were anaesthesized with a final concentration of 0.04% tricaine and the images were obtained with the AxioVision software (version 4.6). The embryos were washed with abundant E3 medium until they recovered.

The embryos were analyzed at 53, 72 and 96 hpf, and the presence of the following phenotypes was searched for in each of the embryos used in the study:
alteration of the somite bonds (somite boundaries).
morphological alterations of the embryos in general.
appearance of edemas in the pericardial area.
morphological alterations in the heart, as well as alterations in blood circulation and alterations in the formation of the vascular system.

B. Results

The main effect produced by MTA is the loss of the posterior cardinal vein (PCV). Since this vein is detected in all the embryos but one at 53 hpf, and since, at 72 hpf, it is absent in half of the embryos, 1 mM MTA would be inhibiting the maintenance and not the formation thereof This causes defects in blood circulation and the accumulation of blood cells in the posterior part of the tail, precisely the area where the PCV is absent and, therefore, blood cannot return to the heart. Pericardial edemas indicative of circulatory defects are also detected, the size whereof is associated with the presence or absence of circulation in the embryo. The embryos which exhibit larger edemas are those wherefrom blood circulation is completely absent. The MTA dose that induces this phenotype is 1 mM. In addition to this phenotype related to the development of the circulatory system, it was detected that 25% of the embryos treated with 1 mM MTA showed a clear dissambling of the somites at 72 hpf and 96 hpf. It was possible to detect some of the somite bonds inside the disorganized tissue, although not most of them. None of these embryos showed defects in the development of the vascular system. Moreover, these embryos showed a moderate ventral curvature of the tail at 53 hpf, which disappeared at later stages.

In some cases, the hearts of the embryos treated with MTA exhibit a tubular morphology, and there is an accumulation of blood cells on the posterior part of the tail, with the presence of pericardial edemas and an alteration or absence of blood circulation.

In addition to other morphological alterations of the embryos following treatment with MTA, such as the slight tail curvature, this molecule also induces disorganisation of the somites, primarily on the most anterior part of the embryos' trunk, in 25% of the embryos. During somitogenesis, the somite segmentation process involves the EMT of the cells that form the bonds between them.

Endocardial cells develop the EMT between 60-72 hpf, and the endocardial cushions begin to appear at 72 hpf, and their formation is completed at 96 hpf. In the zebrafish, the EMT originates the endocardial cushions of the heart's atrio-ventricular (AV) channel from the endocardial epithelium, thereby contributing to the development of the cardiac valve. The determination that the cause of the defects found in the vascular system is the absence of an adequate development of the cardiac valve requires a microscopic-level study of said valve (Beis et al., Development 2005; 132(18): 4193-204).

Therefore, certain phenotypes associated with the inhibition of the EMT process are detected in zebrafish embryos treated with MTA. For example, Notch is also capable of inducing the EMT during cardiac development in zebrafish. We have used a Notch inhibitor (DAPT) as a positive control with the appearance of pericardial edemas, alterations of the somite bonds, and alteration or absence of blood circulation, as previously described (Timmerman et al., Genes Dev 2004; 18(1): 99-115). A similar phenotype appears in zebrafish embryos deficient in the tyrosine phosphatase PEZ protein, a protein that regulates the epithelial-mesenchymal transition. Moreover, said absence causes problems in the maintenance of the somites like those induced by MTA. It is interesting to note that TGFβ₁ is a factor that regulates the PEZ protein in the formation of some of these processes associated with the EMT.

### Example 6

### In Vivo PSC Model: Mouse Deficient in the Mdr2 Gene

The Mdr2 (MDR3 in humans) canalicular phospholipid pump is a member of the superfamily of ABC transporters and the MDR/TAP sub-family. Under physiological conditions, Mdr2 transports phospholipids to the bile and micelles are formed which protect the cholangiocytes from potential damage caused by biliary acids. Mutations in its human ortologue MDR3 cause a wide clinical range of liver disease, from neonatal cholestasis to liver diseases in adults.

Mice deficient in the gene that encodes the Mdr2 protein (KO-Mdr2 mice) exhibit a significant reduction in the biliary production of phospholipids and cholesterol. The lack of phospholipids in the biliary duct of KO-Mdr2 mice may cause the toxic acid bile that damages the biliary duct, ultimately triggering sclerosing cholangitis. A lesion of the biliary epithelium is observed which seems to be due to the toxicity of biliary salts in the absence of a protective effect by phospholipids. The levels of biliary glutathione and cholesterol are lower than normal levels, whereas an increase in the secretion of bilirubin is observed.

These KO-Mdr2 mice -those with the Mdr2 gene deleted- show microscopic and macroscopic characteristics similar to those that occur in human PSC, such as extra- and intrahepatic biliary structures, dilatations and periductal fibrosis. Biliary acids are normally packed in micelles, jointly with phospholipids and cholesterol, in order to protect potentially toxic cholangiocytes from biliary acids, which may cause the necrosis or apoptosis of cholangiocytes. Biliary acids may be capable of inducing a reaction of the phenotype of cholangiocytes characterised by the production of various pro-inflammatory and pro-fibrogenic cytokines and chemokines, as well as the corresponding receptors thereof. Moreover, the KO-Mdr2 mouse is a model for cholestasis (Lammert et al., Hepatology 2004; 39(1): 117-128).

A. Methods

Treatment protocol with MTA in the KO-Mdr2 model. As has been described, these mice spontaneously develop sclerosing cholangitis, due to the lack of the biliary phospholipid transporter (Mdr2 gene, human homologue of MDR3/ABCB4). In the in vivo experiments, MTA was resuspended in saline solution. The administration of MTA (28 mg/kg, every 24 h) was performed for 21 days on the control WT mice and the mice deficient in the Mdr2 gene (KO-Mdr2 mice), 9 weeks of age, when evident symptoms of cholestatic disease attributed to PSC are appreciated. The total RNA was isolated from each liver in order to quantify the expression of the different markers characteristic of the EMT.

B. Results

Hepatomegaly is observed, quantified by an increase in the weight of the liver (left graph) and an increase in the liver weight/total animal weight ratio (right graph). This effect is reversed following treatment with MTA (FIG. 6A).

In the livers of KO-Mdr2 mice, a greater expression of markers indicative of the EMT is observed. The increase in the expression of EMT markers (Tenascin C and TIMP 1) and other cytokines (IL-6) is increased in KO-Mdr2 mice, as quantified by real-time RT-PCR. MTA is capable of reducing the effect on the expression of these markers (FIG. 6B). Following the daily oral administration of MTA, the expression of EMT markers in the total liver is significantly reduced.

### Example 7

### Effects of MTA Following a Prolonged 21-Day Treatment

A. Methods

In order to evaluate hepatic fibrosis, Red Sirium staining was performed which was subsequently quantified. In order to measure the levels of marker enzymes for liver damage, serum was extracted from the WT and KO-Mdr2 mice treated with both carrier (saline solution) and MTA (28 mg/kg), every 24 h for 21 days. These sera were frozen in liquid nitrogen, prior to being stored, at −80° C. The levels of hepatic enzymes (aspartate aminotransferase, AST; Alanine Transaminase, ALT; Alkaline Phosphatase, ALP; Bilirubin) were measured in a Hitachi analyzer (Boehringer Mannheim). The in vivo results representatively summarize at least three independent administration protocols.

Immunohistochemistry. In order to perform histological assays, the liver was fixed in 4% paraformaldehyde for 24 h and sections were prepared in paraffin (4-nm thickness). Stainings with haematoxylin-eosine were performed (data not shown). The stainings of proliferation markers Ki67 in hepatic cells and of marker a-SMA were performed following protocols already described (Fickert et al., Gastroenterology 2006; 130(2): 465-481). The number of positive cells was counted in the ductural periportal areas.

In addition, it was analyzed the Tenascin C expression, an extracellular matrix protein associated to fibrosis.

B. Results

The periodical intake of MTA also significantly improves fibrosis and markers of hepatic cellular damage. In the first place, the livers of the untreated KO-Mdr2 mice showed appreciable signs of fibrosis (visual appearance-elasticity-texture-consistency) at the time of sacrifice, unlike the KO-Mdr2 mice treated with MTA, which indicates the beneficial effect of MTA on the total liver affected by the lack of Mdr2. The graph shows the quantification of fibrosis by the Red Sirium technique, in untreated KO-Mdr2 mice vs mice treated with MTA (FIG. 7A). In the second place, the serum levels of AST, ALT, ALP and bilirubin—which are abnormally high in KO-Mdr2 mice-significantly improve following treatment with MTA (FIG. 7B). On the other hand, the number of cells with positive marking for proliferation marker Ki67 is higher in the periportal fibrous areas. Two representative photographs are shown, and the graph shows that the number of positive cells around the biliary ducts significantly decreases following treatment with MTA (FIG. 7C). Also, we show the marking of α-SMA, which is higher in the KO-Mdr2 mice, decreases following the administration of MTA. The quantification of the expression of α-SMA mRNA by real-time RT-PCR shows that the levels decrease in the liver in the presence of MTA (FIG. 7D).

Finally, we can observe how, after the treatment of the KO-Mdr2 mice over 21 days with MTA, the Tenascin C expression is reverted to the levels present in WT mice (Mdr2+/+) (FIG. 7E).

### Example 8

### Characteristic CsC (Cancer Stem Cells) Markers in KO-Mdr2 Cholangiocytes

KO-Mdr2 mice develop cholangiocarcinoma (epithelial adenoma of the bile conducts) after a few months of life, being the cholangitis the primary risk for the development of the cited neoplasia. A treatment for these patients is not currently available, being surgery the only therapeutic option. Therefore, analyzing the possible presence of the tumor initiating cells in early stages of the disease and being able to eliminated them, would prevent from further complications.

A. Methods

It was analyzed the expression of possible markers of stem cells in WT and KO-Mdr2 cholangiocytes by conventional PCR and real time PCR, said markers were selected from Vimentin, Snaill and Tenascin C. Real time PCR was realized as described in example 3, while the enzyme “Immolase DNA polimerase” was employed for the conventional PCR, the reaction being developed in a conventional thermocycler.

B. Results

We observed that the cholangiocytes from KO-Mdr2 over-express said markers (FIG. 8A) being significantly increased Snaill and Vimentin (FIG. 8B) (Garcion E., et al, Development 2004;20(10):2524-40), (Zhiyong Du., et al, Dig Dis Sci 2010 Epub ahead of print.). On the contrary, the expression of the CK19 ephitelial marker is significantly lower in cholangiocytes from KO-Mdr2 mice compared to cholangiocytes from WT mice.

### Example 9

### MTA Reverses the TGFβ₁ Effect Over the E-Cadherine Expression

E-cadherine is a transmembrane glycoprotein involved in the cellular adhesion of the epithelium. Its minor expression or absence plays an important role in the invasive capacity of the neoplastic cells.

A. Methods

The E-cadherin expression in cholangiocytes was analyzed by an immunofluorescence assay. Cells were seeded over cover slips treated with collagen to improve their adherence. Cells were then treated with 80 pM TGFβ₁ and a 24 h combination of 80 pM TGFβ₁ and 500 μM MTA. Once the cells were mounted, they were incubated with an anti-E-cadherin antibody and the protein expression was observed under a confocal microscope.

B. Results

It was observed the E-cadherin expression at membrane level, in both the cells not treated from WT mice and from KO-Mdr2. Such expression decreases when the cells are treated with TGFβ₁ and when treated with MTA it returns to basal levels. In FIG. 9A are represented the cholangiocytes from KO-Mdr2 mice.

### Example 10

### MTA Reverses the TGFβ₁ Effect Over the Isoform of TGFβ and Collagen Expression/Secretion

One of the CsC described features is that they express TGFβ₁ (Salazar K D., et al, Am J Physiol Lung Cell Mol Physiol 2009;297(5):L1002-11). The over-expression of specific growth factors induces other molecules expression, implicated both in fibrosis (f. ex. collagen) and related to the presence of stem cells (i.e. Tenascin C, involved in both invasion and metastasis).

A. Methods

The expression and medium secretion of TGFβ and collagen was determined by western blot.

Cells were seeded for 48 h, in the presence or absence of 80 pM TGFβ₁ and/or 500 μM_MTA. After the treatment the conditioned media and the cells were collected.

Eight volumes of acetone were added over the collected media and they were incubated over 48 h at −20° C. They were centrifuged and the precipitates containing the proteins were obtained and resuspended in a buffer solution. The TGFβ and collagen expression was analyzed by western blot, as described in example 4. The antibodies employed were the following: anti-TGFβ1, β2, β3 (1:1000, RαD Systems) and anti-collagen, Col A2 (M-80) (1:500, Millipore).

WT mice cholangiocytes were lysed to obtain the proteins and the TGFβ₁ expression was analyzed at intracellular level by western blot.

B. Results

The results obtained by western blot show that the cholangiocytes from KO-Mdr2 mice secrete TGFβ to the media, and that the MTA treatment decreases such secretion (FIG. 10A). On the contrary, the TGFβ expression is only detected at intracellular level in cholangiocytes from WT mice, although the MTA treatment also decreases its expression (FIG. 10B).

The obtained results also show that the collagen secretion is increased when KO-Mdr2 mice cells are treated with TGFβ₁ and the basal levels are reverted when treated with MTA (FIG. 11).

In summary, we can say that, in this model, the KP-Mdr2 mice spontaneously develop sclerosing cholangitis, due to the lack of the biliary phospholipid transporter (Mdr2 gene, human homologue of MDR3/ABCB4). In the livers of the KO-Mdr2 mice, a greater expression of markers indicative of the EMT is observed, which is accompanied by an increase in fibrosis. Following the daily oral administration of MTA, we managed to significantly reduce the expression of EMT markers in the total liver. The periodical intake of MTA also significantly improves the markers for hepatic cellular damage in these mice and the biochemical analysis.

We conclude that the lower presence of cells that have developed the EMT in the hepatic tissue, due to the action of MTA in the established PSC model, improves the pattern of this disease.

1. 5′-methylthioadenosine, its pharmaceutically acceptable salts and/or the prodrugs thereof, for use in the inhibition and/or blockade of the epithelial-mesenchymal transition.

2. 5′-methylthioadenosine, its pharmaceutically acceptable salts and/or the prodrugs thereof, for use according to claim 1, where the epithelial-mesenchymal transition is dependent on TGFβ₁.

3. 5′-methylthioadenosine, its pharmaceutically acceptable salts and/or the prodrugs thereof, for use according to claim 1, in the prevention and/or treatment of a disease associated with the epithelial-mesenchymal transition.

4. 5′-methylthioadenosine, its pharmaceutically acceptable salts and/or the prodrugs thereof, for use according to claim 3, as an adjuvant or additional treatment following a radiotherapy or chemotherapy treatment.

5. 5′-methylthioadenosine, its pharmaceutically acceptable salts and/or the prodrugs thereof, for use according to claim 4, in the elimination of cancer stem cells in subjects who present recurrence in the face of conventional chemotherapeutic agents.

6. 5′-methylthioadenosine, its pharmaceutically acceptable salts and/or the prodrugs thereof, for use according to claim 3, where the disease is an epithelial cancer, a fibrotic disease associated with the EMT or a cholestatic disease.

7. 5′-methylthioadenosine, its pharmaceutically acceptable salts and/or the prodrugs thereof, for use according to claim 6, to prevent the development of metastasis in a subject with epithelial cancer.

8. 5′-methylthioadenosine, its pharmaceutically acceptable salts and/or the prodrugs thereof, for use according to claim 6, where the cholestatic disease is primary sclerosing cholangitis or primary biliary cirrhosis.

9. 5′-methylthioadenosine, its pharmaceutically acceptable salts and/or the prodrugs thereof, for use according to claim 6, where the cholestatic disease is a cholangiocarcinoma.

10. A pharmaceutical composition that comprises:
a) 5′-methylthioadenosine and/or its pharmaceutically acceptable salts and/or the prodrugs thereof, and b) a pharmaceutically acceptable excipient for use in the inhibition and/or blocking of the epithelial-mesenchymal transition.

11. A pharmaceutical composition that comprises:
a) 5′-methylthioadenosine and/or its pharmaceutically acceptable salts and/or the prodrugs thereof, and b) a pharmaceutically acceptable excipient for use according to claim 10, in the prevention and/or treatment of a disease associated with the epithelial-mesenchymal transition.

12. Use of 5′-methylthioadenosine, its pharmaceutically acceptable salts and/or the prodrugs thereof, or of a pharmaceutical composition that comprises a) said 5′-methylthioadenosine, its pharmaceutically acceptable salts and/or the prodrugs thereof, and b) a pharmaceutically acceptable excipient, in the preparation of a medicament for inhibiting and/or blocking the epithelial-mesenchymal transition.

13. Use of 5′-methylthioadenosine, its pharmaceutically acceptable salts and/or the prodrugs thereof, or of a pharmaceutical composition that comprises a) said 5′-methylthioadenosine, its pharmaceutically acceptable salts and/or the prodrugs thereof, and b) a pharmaceutically acceptable excipient, according to claim 12, in the preparation of a medicament for the prevention and/or treatment of a disease associated with the epithelial-mesenchymal transition.

14. A method for the inhibition and/or blocking of the epithelial-mesenchymal transition that comprises administering, to a subject in need thereof, an effective quantity of 5′-methylthioadenosine, its pharmaceutically acceptable salts and/or the prodrugs thereof, or of a pharmaceutical composition that comprises: a) said 5′-methylthioadenosine, its pharmaceutically acceptable salts and/or the prodrugs thereof, and b) a pharmaceutically acceptable excipient.

15. A method according to claim 14, for the prevention and/or treatment of a disease associated with the epithelial-mesenchymal transition.

16. 5′-methylthioadenosine, its pharmaceutically acceptable salts and/or the prodrugs thereof, for use according to claim 2, in the prevention and/or treatment of a disease associated with the epithelial-mesenchymal transition.

17. 5′-methylthioadenosine, its pharmaceutically acceptable salts and/or the prodrugs thereof, for use according to claim 4, where the disease is an epithelial cancer, a fibrotic disease associated with the EMT or a cholestatic disease.

18. 5′-methylthioadenosine, its pharmaceutically acceptable salts and/or the prodrugs thereof, for use according to claim 15, as an adjuvant or additional treatment following a radiotherapy or chemotherapy treatment.

19. 5′-methylthioadenosine, its pharmaceutically acceptable salts and/or the prodrugs thereof, for use according to claim 18, in the elimination of cancer stem cells in subjects who present recurrence in the face of conventional chemotherapeutic agents.

20. 5′-methylthioadenosine, its pharmaceutically acceptable salts and/or the prodrugs thereof, for use according to claim 16, where the disease is an epithelial cancer, a fibrotic disease associated with the EMT or a cholestatic disease.
